SEQUENCE LISTING (10 sequences)

SEQ ID NO: 1 - DNA, 16569 bp, Homo sapiens
  misc_feature (3107)..(3107): n is a, c, g, or t
gatcacaggtctatcaccctattaaccactcacgggagctctccatgcatttggtattttcgtctggggggtatgcacgcgatagcattgcgagacgctggagccggagcaccctatgtcgcagtatctgtctttgattcctgcctcatcctattatttatcgcacctacgttcaatattacaggcgaacatacttactaaagtgtgttaattaattaatgcttgtaggacataataataacaattgaatgtctgcacagccactttccacacagacatcataacaaaaaatttccaccaaaccccccctcccccgcttctggccacagcacttaaacacatctctgccaaaccccaaaaacaaagaaccctaacaccagcctaaccagatttcaaattttatcttttggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaaggaacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacnttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgaaccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggtcataaagcctaaatagcccacacgttccccttaaataagacatcacgatg

SEQ ID NO: 2 - DNA, 23 bp, Artificial Sequence
  Primer
ggtgatagctggttgtccaagat

SEQ ID NO: 3 - DNA, 32 bp, Artificial Sequence
  Primer
cctactatgggtgttaaattttttactctctc

SEQ ID NO: 4 - DNA, 23 bp, Artificial Sequence
  Primer
aatttaactgttagtccaaagag

SEQ ID NO: 5 - DNA, 22 bp, Artificial Sequence
  Primer
gtcaggagatcgagaccatcct

SEQ ID NO: 6 - DNA, 17 bp, Artificial Sequence
  Primer
agtggcgcaatctcggc

SEQ ID NO: 7 - DNA, 25 bp, Artificial Sequence
  Primer
agctactcgggaggctgaggcagga

SEQ ID NO: 8 - DNA, 20 bp, Artificial Sequence
  Primer
ctgttccccaaccttttcct

SEQ ID NO: 9 - DNA, 20 bp, Artificial Sequence
  Primer
ccatgattgtgaggggtagg

SEQ ID NO: 10 - DNA, 18 bp, Artificial Sequence
  Primer
gaccccctaacaaccccc

(19) United States
(12) Patent Application Publication
(10) Pub. No.: US 2017/0107571 A1
(43) Pub. Date: Apr. 20, 2017

(54) QUANTIFICATION OF MITOCHONDRIAL DNA AND METHODS FOR DETERMINING THE QUALITY OF AN EMBRYO

(71) Applicant: CooperSurgical, Inc., Trumbull, CT (US)

(72) Inventors: Dagan Wells, Oxford (GB); Elpida Fragouli, Oxford (GB); Santiago Munne, Short Hills, NJ (US)

(21) Appl. No.: 15/171,042

(22) Filed: Jun. 2, 2016

Related U.S. Application Data
(60) Provisional application No. 62/242,460, filed on Oct. 16, 2015.

(51) Int. Cl. C12Q 1/68

(52) U.S. Cl. CPC C12Q 1/6876; C12Q 2600/158; C12Q 2600/16

(57) ABSTRACT

Materials and methods provided herein are useful for determining an implantation threshold for a euploid embryo and for determining the potential of a euploid embryo to implant and initiate a pregnancy.

### CROSS-REFERENCE TO RELATED APPLICATIONS

This application claims priority to U.S. Provisional Patent Application No. 62/242,460, filed on Oct. 16, 2015, the entire contents of which is incorporated herein by reference.

### TECHNICAL FIELD

The present disclosure relates generally to the fields of reproductive medicine. More specifically, this disclosure relates to noninvasive methods and kits for determining the potential of an embryo to implant and initiate a pregnancy.

### BACKGROUND

The selection of embryos with higher implantation potential is one of the major challenges in assisted reproductive technology such as In vitro fertilization (IVF). IVF involves combining eggs and sperm outside the body in a laboratory to form an embryo. Once an embryo forms, it is implanted in the uterus where it will develop further. One challenge facing IVF techniques is low success rates of embryonic implantation and pregnancy. As such, there is a need to better understand the mechanisms affecting proper embryo development, which will in turn allow the development of tools for identifying embryos having high implantation potential, and for increasing the rates of successful pregnancy. By selecting embryos that possess the optimal implantation potential, the odds of successful in vitro fertilization procedures improve. Considering the costs of IVF, this could result in savings of thousands or tens of thousands of dollars as the procedure may not need to be repeated as often before resulting in a successful pregnancy.

Thus, in order to improve the efficiency of assisted reproductive treatments, superior methods for the identification of viable embryos are urgently required. The screening of embryos for cytogenetic abnormalities prior to transfer to the uterus allows the main cause of embryonic failure (i.e. aneuploidy) to be avoided. However, even the transfer of a morphologically ‘perfect’ embryo, which is additionally considered chromosomally normal following analysis of biopsied cells, cannot guarantee the initiation of a successful pregnancy (only about two thirds of such embryos actually produce a child). It is clear that additional elements play a role in embryo viability. Important factors might conceivably include mitochondrial number/capacity and accompanying effects on ATP content and/or metabolic activity [17].

Mitochondria play a vital role in embryo development. They are the principal site of energy production and have various other critical cellular functions. Mitochondria are involved in the regulation of multiple essential cellular processes, such as apoptosis, amino acid synthesis, calcium homeostasis, and the generation of energy in the form of ATP via the process of oxidative phosphorylation (OXPHOS) [1-5]. For this reason mitochondria are considered as the principal cellular power houses. They are unique compared to other organelles in animal cells in that they contain one or more copies of their own genome. Despite the importance of this organelle, little is known about the extent of variation in mtDNA between individual human embryos prior to implantation, or the association between the relative amount of mtDNA and the ability of the human embryo to implant into the uterus.

### SUMMARY

The present disclosure is based in part on the unexpected discovery that the relative amount of mtDNA in a embryo (e.g., a human embryo) is predictive of the ability of the embryo to implant into the uterus. Thus, provided herein are materials and methods for determining an implantation threshold for an embryo and for determining the implantation potential of an embryo (e.g., a euploid embryo) based on the relative amount of mtDNA found in the embryo. The materials and methods provided herein overcome limitations associated with known assays for mitochondrial DNA (mtDNA) quantification. The inventive methods described herein exhibit high sensitivity and specificity.

As demonstrated herein, there is a correlation between mtDNA quantity and the implantation potential of an embryo. The assessment of mtDNA quantity can be used to identify embryos having the highest potential for implantation leading to healthy pregnancies.

In one aspect, the disclosure provide a method for determining the relative quantity of mitochondrial DNA in an embryo, which comprises providing a DNA sample obtained from the embryo, determining an amount of mitochondrial DNA (mtDNA) in the DNA sample, determining an amount of a reference DNA in the DNA sample, and comparing the amount of mtDNA to the amount of reference DNA to determine a relative quantity of mtDNA in the embryo. The relative quantity of mtDNA in the embryo provides a reliable indicator of the an implantation potential of the embryo.

In one aspect, the disclosure provides a method for determining an implantation potential of an embryo, the method comprising providing a DNA sample obtained from the embryo, determining an amount of mitochondrial DNA (mtDNA) in the DNA sample, determining an amount of a reference DNA in the DNA sample, and comparing the amount of mtDNA to the amount of reference DNA to determine a relative quantity of mtDNA in the embryo, wherein the relative quantity of mtDNA in the embryo is indicative of the implantation potential of the embryo. In some embodiments the embryo is a euploid embryo.

Determining the amount of mtDNA in the DNA sample and determining the amount of a reference DNA in the DNA sample can be performed using quantitative PCR, such as, for example, real time PCR.

In some embodiments of the methods disclosed herein, a DNA sample is obtained from an embryo 1, 2, 3, 4, 5, 6 or 7 days post implantation. For example, the DNA sample is obtained from an embryo or 1-3 days, 2-4 days, 3-5 days, 5-7 days, 1-7 days, or 4-7 days post fertilization.

In some embodiments, determining the amount of mtDNA in the DNA sample comprises amplifying the mtDNA by contacting the DNA sample with a primer pair comprising SEQ ID NOs: 2 and 3 targeting a sequence transcribing 16S to produce a 16S amplicon or a primer pair comprising SEQ ID NOs: 8 and 9 targeting a sequence encoding NADH-ubiquinone oxidoreductase chain 4 (MT-ND4) to produce a MT-ND4 amplicon. The method may further comprise detecting the 16S amplicon by contacting the DNA sample with a probe comprising SEQ ID NO: 4 targeting a sequence within the 16S amplicon, and/or detecting the MT-ND4 amplicon by contacting the DNA sample with a probe comprising SEQ ID NO: 10 targeting a sequence within the MT-ND4 amplicon.

In some embodiments, determining the amount of a reference DNA in the DNA sample comprises amplifying the reference DNA by contacting the DNA sample with a primer pair comprising SEQ ID NOs: 5 and 6 targeting an Alu sequence to produce an Alu amplicon. The method may further comprise detecting the Alu amplicon by contacting the DNA sample with a probe comprising SEQ ID NO: 7 targeting a sequence within the Alu amplicon.

In some embodiments, determining the amount of a reference DNA in a DNA sample comprises amplifying the reference DNA by contacting the DNA sample with a primer pair targeting a repetitive DNA sequence or a multicopy sequence (e.g. the L1 repeat or an Alu sequence) to produce a target amplicon. The method may further comprise detecting the target amplicon by contacting the DNA sample with a probe targeting a sequence within the amplicon. The repetitive DNA sequence or multicopy sequences targeted in the embodiments described herein are known in the art. In some embodiments, determining the amount of a reference DNA in a DNA sample comprises amplifying at least one (e.g., 1, 2, 3, 4, 5, 6, 7, 8, 9, or 10, etc.)

In some aspects, the methods disclosed herein comprise comparing the relative quantity of mtDNA in the euploid embryo to an implantation potential threshold, wherein a relative quantity of mtDNA in the embryo below the implantation potential threshold is indicative of a favorable implantation potential of the euploid embryo and a relative quantity of mtDNA in the embryo exceeding the implantation potential threshold is indicative of an unfavorable implantation potential of the euploid embryo.

In one aspect, the disclosure provides a method for selecting an embryo for implantation, the method comprising determining the relative amount of mitochondrial DNA in an embryo sample compared to a reference nucleic acid sequence in the embryo, wherein determining comprises preparing a reaction mix comprising i) a nucleic acid sample from an embryo; ii) a first synthetic oligonucleotide primer pair directed against a first mitochondrial DNA sequence; and iii) a reference synthetic oligonucleotide primer pair directed against a of reference target nucleic acid sequence, amplifying the reaction mixture to produce a first mitochondrial DNA sequence product and a reference target nucleic acid sequence product; assessing the amount of the amplified first mitochondrial DNA sequence product compared with the amount of the amplified reference target nucleic acid sequence product, and selecting an embryo for implantation when the measured amount of the amplified first mitochondrial DNA sequence product relative to the amplified reference target nucleic acid sequence product is below an implantation potential threshold value. The reference nucleic acid sequence can be a chromosomal nucleic acid sequence.

The reaction mix may comprise a second synthetic oligonucleotide primer pair directed against a second mitochondrial DNA sequence. In some embodiments, the reaction mix may comprise a third synthetic oligonucleotide primer pair directed against a third mitochondrial DNA sequence In some embodiments, the reaction mix may comprise a fourth synthetic oligonucleotide primer pair directed against a fourth mitochondrial DNA sequence. In some embodiments, the reaction mix may comprise five or more (e.g., 5, 6, 7, 8, 9, or 10, etc.) synthetic oligonucleotide primer pairs, directed against five or more (e.g., 5, 6, 7, 8, 9, or 10, etc.) mitochondrial DNA sequences.

In some embodiments, the methods comprise amplifying the reaction mixture to produce a first mitochondrial DNA sequence product, a second mitochondrial DNA sequence product, and a reference chromosomal target nucleic acid sequence product; and measuring the amount of each amplified product; comparing the measured amount of the amplified first mitochondrial DNA sequence product and the amplified second mitochondrial DNA sequence product with the amplified reference chromosomal target nucleic acid sequence product; and selecting an embryo for implantation when the measured amount of the amplified first mitochondrial DNA sequence product and the amplified second mitochondrial DNA sequence product relative to the amplified reference chromosomal target nucleic acid sequence product are below an implantation potential threshold value. In some embodiments, the reaction mix may comprise a third synthetic oligonucleotide primer pair directed against a third mitochondrial DNA sequence.

In one aspect, the disclosure provides a method for selecting an embryo for implantation, the method comprising measuring the relative amount of mitochondrial DNA in an embryo sample compared to a reference nucleic acid sample, wherein measuring comprises preparing a reaction mix comprising i) a nucleic acid sample from an embryo; ii) a first synthetic oligonucleotide primer pair directed against a first mitochondrial DNA sequence; iii) a second synthetic oligonucleotide primer pair directed against a second mitochondrial DNA sequence; and iv) a reference synthetic oligonucleotide primer pair directed against a reference chromosomal target nucleic acid sequence, amplifying the reaction mixture to produce a first mitochondrial DNA sequence product, a second mitochondrial DNA sequence product, and a reference chromosomal target nucleic acid sequence product; and measuring the amount of each amplified product; comparing the measured amount of the first mitochondrial DNA sequence product and the second mitochondrial DNA sequence product with the reference chromosomal target nucleic acid sequence product; and selecting an embryo for implantation when the measured amount of the first mitochondrial DNA sequence product and the second mitochondrial DNA sequence product relative to the reference chromosomal target nucleic acid sequence product are below an implantation potential threshold value. In some embodiments, embryos are identified as not being suitable for implantation when the amount of the first mitochondrial DNA sequence product and the second mitochondrial DNA sequence product relative to the reference chromosomal target nucleic acid sequence product are above an implantation potential threshold value.

In some embodiments, the synthetic oligonucleotide primer pairs targeting mtDNA (e.g., the first or second) are directed against a mitochondrial DNA sequence selected from the group of a mitochondrial DNA sequence encoding a 12S RNA, a mitochondrial DNA sequence encoding a 16S RNA, a mitochondrial DNA sequence encoding NADH dehydrogenase subunit 1 (MT-ND1), a mitochondrial DNA sequence NADH dehydrogenase subunit 1 (MT-ND1), a mitochondrial DNA sequence encoding NADH dehydrogenase subunit 2 (MT-ND2), a mitochondrial DNA sequence encoding NADH dehydrogenase subunit 3 (MT-ND3), a mitochondrial DNA sequence encoding NADH dehydrogenase subunit 4 (MT-ND4), a mitochondrial DNA sequence encoding NADH dehydrogenase subunit 5 (MT-ND5), a mitochondrial DNA sequence encoding Cytochrome b, mitochondrial Cytochrome c oxidase subunit 1, 2 or 3, and a mitochondrial DNA sequence encoding an ATP synthase.

In some embodiments, the first synthetic oligonucleotide primer pair is directed against the mitochondrial DNA sequence encoding the 16S rRNA gene sequence. For example, the first synthetic oligonucleotide primer pair comprises a primer having at least 70% sequence identity to a nucleic acid sequence of SEQ ID NO: 2 and a primer having at least 70% sequence identity to a nucleic acid sequence of SEQ ID NO: 3.

In some embodiments, the second synthetic oligonucleotide primer pair is directed against the mitochondrial DNA sequence encoding the NADH-ubiquinone oxidoreductase chain 4 (MT-ND4) gene sequence. For example, the second synthetic oligonucleotide primer pair comprises a primer having at least 70% sequence identity to a nucleic acid sequence of SEQ ID NO: 8 and a primer having at least 70% sequence identity to a nucleic acid sequence of SEQ ID NO: 9.

In some embodiments, the reference synthetic oligonucleotide primer pair is directed against the Alu sequence, the L1 sequence, glyceraldehyde 3-phosphate dehydrogenase (GAPDH), or β-actin (ActB). In some embodiments, the reference synthetic oligonucleotide primer pair is directed against the Alu sequence. For example, the reference synthetic oligonucleotide primer pair comprises a primer having at least 70% sequence identity to a nucleic acid sequence of SEQ ID NO: 5 and a primer having at least 70% sequence identity to a nucleic acid sequence of SEQ ID NO: 6.

In some embodiments, the amplifying step is preformed using a quantitative or semi-quantitative RT-PCR method.

In one aspect, the disclosure provides a kit for determining the implantation potential of an embryo, the kit comprising a first synthetic oligonucleotide primer pair directed against a first mitochondrial DNA sequence and a second synthetic oligonucleotide primer pair directed against a reference gene sequence DNA. The reference gene sequence can be chromosomal gene sequence. In some embodiments, the first synthetic oligonucleotide primer pair is directed to a mitochondrial DNA sequence selected from a mitochondrial DNA sequence encoding a 12S RNA, a mitochondrial DNA sequence encoding a 16S RNA, a mitochondrial DNA sequence encoding NADH dehydrogenase subunit 1 (MT-ND1), a mitochondrial DNA sequence NADH dehydrogenase subunit 1 (MT-ND1), a mitochondrial DNA sequence encoding NADH dehydrogenase subunit 2 (MT-ND2), a mitochondrial DNA sequence encoding NADH dehydrogenase subunit 3 (MT-ND3), a mitochondrial DNA sequence encoding NADH dehydrogenase subunit 4 (MT-ND4), a mitochondrial DNA sequence encoding NADH dehydrogenase subunit 5 (MT-ND5), a mitochondrial DNA sequence encoding Cytochrome b, mitochondrial Cytochrome c oxidase subunit 1, 2 or 3, and a mitochondrial DNA sequence encoding an ATP synthase.

In some embodiments, the kit may comprise a primer pair comprising SEQ ID NOs: 2 and 3 targeting a sequence transcribing 16S or a primer pair of SEQ ID NOs: 8 and 9 targeting a sequence encoding NADH-ubiquinone oxidoreductase chain 4 (MT-ND4) to produce a MT-ND4 amplicon. The kit may further comprise a probe comprising SEQ ID NO: 4 targeting a sequence within the 16S amplicon, and/or a probe of SEQ ID NO: 10 targeting a sequence within the MT-ND4 amplicon.

In some embodiments, the kit comprises a reference synthetic oligonucleotide having a sequence of SEQ ID NOs: 5 and/or 6 targeting an Alu sequence to produce an Alu amplicon. The kit may further comprise a probe comprising SEQ ID NO: 7 targeting a sequence within the Alu amplicon.

In some embodiments, the kit comprises a reference synthetic oligonucleotide primer pair directed against the Alu sequence, the L1 sequence, glyceraldehyde 3-phosphate dehydrogenase (GAPDH), or β-actin (ActB). For example, the reference synthetic oligonucleotide primer pair can be directed against the Alu sequence.

In some embodiments, the kit comprises a reference synthetic oligonucleotide having a sequence having at least 70% sequence identity to a nucleic acid sequence of SEQ ID NO: 5 and primer having at least 70% sequence identity to a nucleic acid sequence of SEQ ID NO: 6. The kit may also comprise a probe directed against the Alu sequence, wherein the probe comprises SEQ ID NO: 7.

In some embodiments, the kit comprises one or more of deoxynucleotides (dNTPs), a DNA polymerase, e.g., a thermostable DNA polymerase such as, for example a Taq DNA polymerase or an AmpliTaq® DNA polymerase, and a buffer, such as a Tris-EDTA (TE) buffer. The kit can contain a positive control DNA sample and a negative control DNA sample. The kit can also contain a third synthetic oligonucleotide primer pair directed against a second mitochondrial DNA sequence.

In one aspect, the disclosure provides a method for determining an implantation potential threshold of an embryo, the method comprising determining the amount of mitochondrial DNA (mtDNA) in one or more DNA samples obtained from implanting euploid embryos, determining the amount of mtDNA in one or more DNA samples obtained from non-implanting euploid embryos, and comparing the amount of mtDNA obtained from the implanting euploid embryos with the amount of mtDNA obtained from the non-implanting embyros, identifying an implantation potential threshold value by determining the relative amount of mtDNA from implanting euploid embryos compared with the amount of mtDNA obtained from the implanting euploid embryos. Determining the amount of mtDNA in a DNA sample is performed using quantitative PCR, such as real time PCR.

In some embodiments, determining the amount of mtDNA in the first DNA sample comprises amplifying the mtDNA in the sample by contacting the DNA sample with a primer pair comprising SEQ ID NOs: 2 and 3 targeting a sequence transcribing 16S to produce a 16S amplicon or a primer pair comprising SEQ ID NOs: 8 and 9 targeting a sequence encoding NADH-ubiquinone oxidoreductase chain 4 (MT-ND4) to produce a MT-ND4 amplicon, and amplifying a reference DNA sequence by contacting the DNA sample with a primer pair comprising SEQ ID NOs: 5 and 6 targeting an Alu sequence to produce an Alu amplicon.

In some embodiments, the methods comprise detecting the 16S amplicon by contacting the DNA sample with a probe comprising SEQ ID NO: 4 targeting a sequence within the 16S amplicon, and/or detecting the Alu amplicon by contacting the DNA sample with a probe comprising SEQ ID NO: 7 targeting a sequence within the Alu amplicon.

In some embodiments, the methods comprise detecting the MT-ND4 amplicon by contacting the DNA sample with a probe comprising SEQ ID NO: 10 targeting a sequence within the MT-ND4 amplicon, and/or detecting the Alu amplicon by contacting the DNA sample with a probe comprising SEQ ID NO: 7 targeting a sequence within the Alu amplicon.

In one aspect, the disclosure provides a method for determining the relative quantity of mitochondrial DNA in an embryo, wherein the method includes providing a DNA sample obtained from the embryo; determining an amount of mitochondrial DNA (mtDNA) in the DNA sample; determining an amount of a reference DNA in the DNA sample; and comparing the amount of mtDNA to the amount of reference DNA to determine a relative quantity of mtDNA in the embryo. In some embodiments of all aspects the relative quantity of mtDNA in the embryo is indicative of an implantation potential of the embryo.

In some embodiments of all aspects determining the amount of mtDNA in the DNA sample and the determining the amount of a reference DNA in the DNA sample include quantitative PCR. In some embodiments of all aspects, the quantitative PCR comprises real time PCR.

In some embodiments of all aspects, a DNA sample is obtained from the embryo 1, 2, 3, 4, 5, 6 or 7 days posit implantation, or 1-3 days, 2-4 days, 3-5 days, 5-7 days, 1-7 days, or 4-7 days post fertilization. In some embodiments, the embryo is a euploid embryo.

In some embodiments, determining the amount of mtDNA in the DNA sample comprises amplifying the mtDNA by contacting the DNA sample with a primer pair comprising SEQ ID NOs: 2 and 3 targeting a sequence transcribing 16S to produce a 16S amplicon or a primer pair comprising SEQ ID NOs: 8 and 9 targeting a sequence encoding NADH-ubiquinone oxidoreductase chain 4 (MT-ND4) to produce a MT-ND4 amplicon. In some embodiments, the method further includes detecting the 16S amplicon by contacting the DNA sample with a probe comprising SEQ ID NO: 4 targeting a sequence within the 16S amplicon. In some embodiments, the method further includes detecting the MT-ND4 amplicon by contacting the DNA sample with a probe comprising SEQ ID NO: 10 targeting a sequence within the MT-ND4 amplicon.

In some embodiments, determining the amount of a reference DNA in the DNA sample comprises amplifying the reference DNA by contacting the DNA sample with a primer pair comprising SEQ ID NOs: 5 and 6 targeting an Alu sequence to produce an Alu amplicon.

In some embodiments, the method further includes detecting the Alu amplicon by contacting the DNA sample with a probe comprising SEQ ID NO: 7 targeting a sequence within the Alu amplicon.

Some embodiments further include comparing the relative quantity of mtDNA in the euploid embryo to an implantation potential threshold; wherein a relative quantity of mtDNA in the embryo below the implantation potential threshold is indicative of a favorable implantation potential of the euploid embryo and a relative quantity of mtDNA in the embryo exceeding the implantation potential threshold is indicative of an unfavorable implantation potential of the euploid embryo.

In one aspect, the disclosure provides a method that includes determining the amount of mitochondrial DNA (mtDNA) in one or more DNA samples obtained from implanting euploid embryos; determining the amount of mtDNA in one or more DNA samples obtained from non-implanting euploid embryos; and calculating an implantation potential threshold value by comparing the amount of mtDNA from implanting euploid embryos with the amount of mtDNA obtained from the non-implanting euploid embryos.

In some embodiments, determining the amount of mtDNA in a DNA sample is performed using quantitative PCR. In some embodiments, the quantitative PCR comprises real time PCR.

In some embodiments, determining the amount of mtDNA in the first DNA sample includes: amplifying the mtDNA in the sample by contacting the DNA sample with a primer pair comprising SEQ ID NOs: 2 and 3 targeting a sequence transcribing 16S to produce a 16S amplicon or a primer pair comprising SEQ ID NOs: 8 and 9 targeting a sequence encoding NADH-ubiquinone oxidoreductase chain 4 (MT-ND4) to produce a MT-ND4 amplicon; and amplifying a reference DNA sequence by contacting the DNA sample with a primer pair comprising SEQ ID NOs: 5 and 6 targeting an Alu sequence to produce an Alu amplicon.

In some embodiments, the method further includes: detecting the 16S amplicon by contacting the DNA sample with a probe comprising SEQ ID NO: 4 targeting a sequence within the 16S amplicon; and detecting the Alu amplicon by contacting the DNA sample with a probe comprising SEQ ID NO: 7 targeting a sequence within the Alu amplicon.

In some embodiments, the method also includes: detecting the MT-ND4 amplicon by contacting the DNA sample with a probe comprising SEQ ID NO: 10 targeting a sequence within the MT-ND4 amplicon; and detecting the Alu amplicon by contacting the DNA sample with a probe comprising SEQ ID NO: 7 targeting a sequence within the Alu amplicon.

In one aspect, the disclosure provides a method for determining the implantation potential of an embryo, the method includes determining the relative amount of mitochondrial DNA in an embryo sample compared to a reference nucleic acid sample, wherein determining comprises preparing a reaction mix comprising i) a nucleic acid sample from an embryo; ii) a first synthetic oligonucleotide primer pair directed against a first mitochondrial DNA sequence; and iii) a reference synthetic oligonucleotide primer pair directed against a of reference chromosomal target nucleic acid sequence; amplifying the reaction mixture to produce a first mitochondrial DNA sequence product and a reference chromosomal target nucleic acid sequence product; and assessing the amount of the amplified first mitochondrial DNA sequence product compared with the amount of the amplified reference chromosomal target nucleic acid sequence product, wherein the relative quantity of mtDNA in the embryo is indicative of the implantation potential of the embryo.

In some embodiments of all methods, determining the relative amount of mitochondrial DNA further comprises: iv) a second synthetic oligonucleotide primer pair directed against a second mitochondrial DNA sequence; and the amplifying step further comprises amplifying the reaction mixture to produce a second mitochondrial DNA sequence product; and the assessing step further comprises assessing the amount of the amplified second mitochondrial DNA sequence product with the amount of the amplified reference chromosomal target nucleic acid sequence product, wherein the relative quantity of the first and the second mitochondrial DNA sequence products are indicative of the implantation potential of the embryo.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a map of human mitochondrial DNA.

FIG. 2 provides a series of graphs showing the relationship between mtDNA quantity, female age and embryo chromosome constitution. A) Data obtained during quantitative real-time PCR analysis of TE samples removed from 302 blastocysts demonstrated a statistically significant increase (P=0.003) in the level of mtDNA in relation to advancing female age. This phenomenon was evident for both euploid and aneuploid blastocysts. B) Real-time PCR analysis of 39 blastomeres showed that cleavage stage embryos from reproductively younger women contained significantly (P=0.01) higher mtDNA levels, compared to those generated by reproductively older women. C) Real-time PCR analysis of TE samples also demonstrated that aneuploid blastocysts (n=99) contained significantly (P=0.025) larger quantities of mtDNA at all ages, compared to those that were euploid (n=203). Statistical analysis of mtDNA values took place with the use of unpaired two-tailed t-tests.

FIG. 3 is a graph showing mtDNA quantification via NGS analysis of chromosomally normal and abnormal blastocysts. NGS analysis of TE samples biopsied from 38 embryos showed a statistically significant increase (P=0.006) in the mtDNA levels occurring in the presence of chromosome errors.

FIG. 4 is a graph showing the mtDNA content of chromosomally normal blastocysts in relation to clinical outcome. On average, chromosomally normal blastocysts capable of establishing a clinical pregnancy contained significantly (P=0.007) lower levels of mtDNA compared to chromosomally normal blastocysts that failed to do so.

FIGS. 5A-5C are a series of graphs showing blastocyst mtDNA quantity threshold in relation to clinical outcome. 5A) The mtDNA quantity viability threshold for euploid blastocysts, established via retrospective analysis of TE biopsies from transferred embryos with known outcomes. All blastocysts producing viable pregnancies contained mtDNA quantities below the 0.003 value (red line) whereas mtDNA quantities above this value were associated with failure to achieve an ongoing clinical pregnancy. 5B) Results of the prospective blinded study. The mtDNA threshold used was the same as that established in the retrospective study (A). Validity was confirmed, since all blastocysts producing viable pregnancies contained mtDNA quantities below the cut-off (red line) and no blastocysts with mtDNA quantities above this value achieved an ongoing clinical pregnancy. 5C) NGS analysis of the mtDNA level in 23 euploid TE samples. The corresponding embryos were transferred during SET cycles, and clinical outcomes were known for 21 of them. As with the real-time PCR experiments, mtDNA levels were lower in the seven implanting embryos (note- the y-axis scale is different for NGS analyses and consequently cut-off values differ).

### DETAILED DESCRIPTION

Provided herein are materials and methods for determining the potential of an embryo (e.g., a euploid embryo) to implant in the uterus (i.e., the “implantation potential”) and initiate a pregnancy. As demonstrated herein, there is a correlation between mitochondrial DNA (mtDNA) quantity and the implantation potential of an embryo. The assessment of mtDNA quantity can be used to identify embryos with the highest ability to lead to healthy pregnancies and live births. Thus, in some aspects, the present disclosure provides compositions and methods for the quantification of mitochondrial DNA (mtDNA) in a preimplantation, i.e., germinal stage, embryo (e.g., a zygote or a blastocyst) for use in determining the implantation potential of the embryo. For example, provided herein are primers and probes that can be used in quantitative PCR methods (e.g., real time PCR) to determine the implantation potential of an embryo.

The term “embryo” refers to a fertilized oocyte or zygote. Said fertilization may intervene under a classical in vitro fertilization (cIVF) or under an intracytoplasmic sperm injection (ICSI) protocol.

In one aspect, the disclosure provides methods for selecting an embryo for implantation, the method comprising: determining the amount of mitochondrial DNA in an embryo sample compared to a reference nucleic acid sequence in the embryo sample, and selecting an embryo for implantation when the determined amount of mitochondrial DNA is increased compared to the determined amount of the reference nucleic acid sample in the embryo.

The terms “increased”, “increase” or “up-regulated” are all used herein to generally mean an increase by a statistically significant amount; for the avoidance of any doubt, the terms “increased” or “increase” means an increase of at least about 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold increase, or any increase between 1.0-fold and 10-fold or greater as compared to a reference level. In some embodiments, the reference level is the level of a reference nucleic acid sequence in the embryo sample.

The terms “decrease”, “decreased”, “reduced”, “reduction” or “down-regulated” are all used herein generally to mean a decrease by a statistically significant amount. However, for avoidance of doubt, “reduced”, “reduction”, “down-regulated” “decreased” or “decrease” means a decrease by at least about 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold decrease, or any decrease between 1.0-fold and 10-fold or greater as compared to a reference level. In some embodiments, the reference level is the level of a reference nucleic acid sequence in the embryo sample.

One skilled in the art will appreciate that minor changes can be made to any of the nucleic acid molecules described herein (e.g., primers and/or probes), and the variant nucleic acid molecules can be used in the methods provided herein, such as a nucleic acid molecule having at least or about 90%, at least 95%, at least 97%, at least 98%, at least 99% or 100% sequence identity to the nucleic acid molecules described herein, e.g., any of SEQ ID NOs: 2-10.

One skilled in the art will recognize that the nucleic acid molecules described herein (e.g., primers and/or probes) can be obtained by standard molecular biology techniques described in Current Protocols in Molecular Biology (1999 Ausubel et al. (editors) John Wiley & Sons, Inc.) or by chemical synthesis or by nucleic acid analogs.

### Mitochondrial DNA

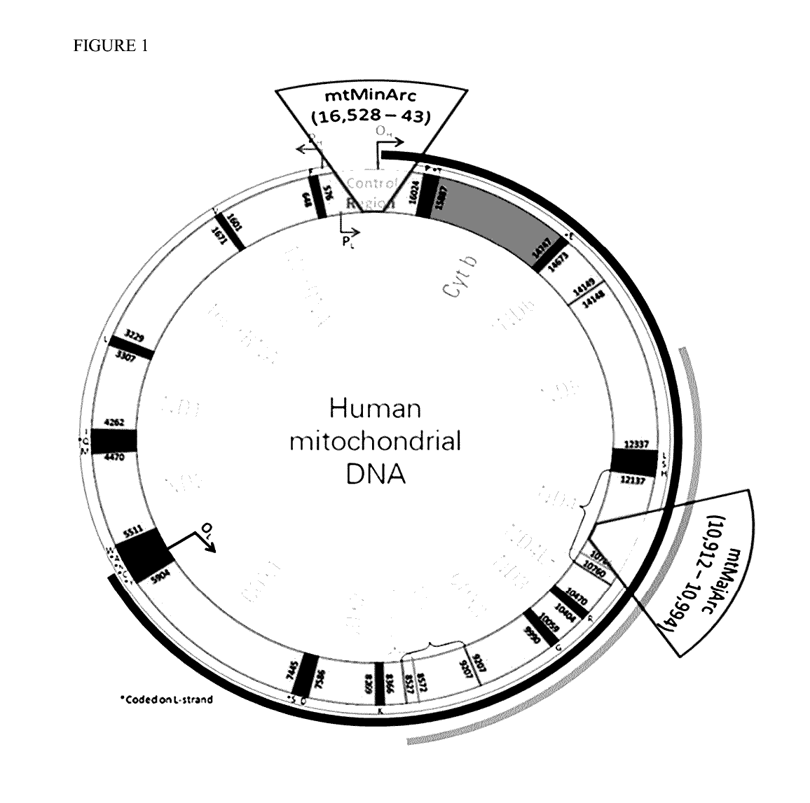

The mitochondrial DNA (mtDNA) is circular and composed of 16.6 kb of double stranded DNA. (FIG. 1) Genes encoded by this DNA molecule have direct roles in cellular metabolism, producing subunits of several complexes with key roles within the electron transport chain (ETC) [6]. Complexes encoded by the mitochondrial genome, along with other ETC components, are situated in the inner mitochondrial membrane and are vital for the production of ATP in the cell. Additionally, mtDNA encodes some of the components of the organelle's transcriptional and translational machinery including 22 tRNAs and 2 rRNAs, with the remainder being encoded by the nuclear genome [6]. It has been shown that cells are capable of redistributing their mitochondria so as to replace damaged organelles, and adjust to variation in intracellular energy requirements [7].

The mitochondrial content of mammalian cells ranges from a few hundred to thousands, determined by the cell's volume and energy needs. The human mature oocyte is among the cell types with the highest content for both mitochondria and mtDNA [1]. Oocyte mitochondrial replication begins during fetal development with cells of the oogonia containing approximately 200 mitochondria [reviewed in 8]. Replication continues in synchrony with maturation, so that just before fertilization an oocyte arrested at metaphase II contains approximately 100,000 mitochondria and between 50,000 and 550,000 copies of the mtDNA [1, 9-13].

Mammalian embryos inherit mitochondria (and thus mtDNA) exclusively from the population found in the oocyte just prior to fertilization. Data from quantification of mtDNA in human cleavage stage embryos suggests that amounts remain stable during the first three days of preimplantation development [1, 12-16]. Significant replication of mtDNA is not thought to be initiated until after the embryo has undergone the first cellular differentiation into trophectoderm (TE) and inner cell mass and has become a blastocyst [3, 8].

Preimplantation development is a dynamic and energy demanding process during which mitochondrial functions are critical. Early embryos require adequate energy levels so that they can successfully progress through each cell division. Existing data suggest that correct oocyte mitochondrial function and mtDNA gene expression are essential during these early stages of life. Specifically, an association has been shown between the ATP content of human oocytes, the developmental potential of an embryo, and the outcome of an IVF cycle [17].

Since mitochondrial functions are critical during the first few days of life, the inventors performed a thorough investigation of mtDNA in human preimplantation embryos which had successfully reached the blastocyst stage of development. Specifically, the inventors examined the relationship between human blastocyst mtDNA content, female patient age, embryo chromosome status, viability and implantation potential. Additionally, we attempted to shed light on the stage of preimplantation development during which mtDNA replication is first up-regulated, with the potential to increase the mtDNA content of individual cells. As well as relative quantification of mtDNA, a detailed analysis of the mitochondrial genome was undertaken, searching for mutations, deletions and polymorphisms.

In some embodiments, the embryo is a euploid embryo. As shown herein, quantification of mtDNA obtained from implanting and non-implanting euploid blastocysts can be used to establish a threshold. The threshold is a value which allows a clinician to determine the implantation potential of a euploid embryo. For example, a mtDNA quantity which falls below the threshold indicates a favorable implantation potential of an embryo while a mtDNA quantity which exceeds the threshold indicates an unfavorable implantation potential of an embryo.

In relative quantification, the amount of the target DNA (e.g., mtDNA) is detected and normalized to the amount of a reference DNA in a single sample to determine a relative amount of the target DNA. Analysis of the relative amount of mtDNA allows the establishment of a threshold to determine the potential of an embryo to implant (i.e., the “implantation potential”) and initiate a pregnancy. As described in herein, a relative amount of mtDNA can be determined by many means including, but not limited to, real-time PCR or next generation sequencing (NGS).

This threshold can be established by analyzing the mitochondrial quantities present in samples with known pregnancy outcomes. In some embodiments, one or more samples associated with embryos having positive and negative pregnancy outcomes are analyzed to determine the threshold. A standard curve and absolute quantification could be employed, but is not a requirement. It will be understood by a person of skill in the art that the threshold for determining the implantation potential of an embryo may require optimization based on a number of variables including, but not limited to, the assay sensitivity, the technician performing the assay, and/or the quantity/quality of the reference DNA.

In some embodiments, a relative quantity of mtDNA is determined by real-time PCR, such as quantitative PCR and the threshold relative mtDNA quantity to determine implantation is 0.003. A preimplantation embryo having a relative mtDNA quantity less than about 0.003 is predicted to implant. For example, a preimplantation embryo predicted to implant can have a relative mtDNA quantity of about 0.0029, about 0.0025, about 0.002, about 0.0015, about 0.001, about 0.0008, about 0.0005, about 0.0003, about 0.0002, about 0.0001, about 0.00008, or about 0.00005, about 0.00004, about 0.00003, or about 0.00002. In some embodiments, a preimplantation embryo predicted to implant has a relative mtDNA quantity less than 0.002. In some embodiments, a preimplantation embryo predicted to implant has a relative mtDNA quantity less than 0.001.

A preimplantation embryo having a relative mtDNA quantity greater than about 0.003 is unable to implant. For example, a preimplantation embryo predicted to not implant can have a relative mtDNA quantity of about 0.0031, about 0.0035, about 0.004, about 0.0045, about 0.005, about 0.006, about 0.007, about 0.008, about 0.009, about 0.01, or about 0.02. In some embodiments, a preimplantation embryo predicted to implant has a relative mtDNA quantity greater than about 0.004. In some embodiments, a preimplantation embryo predicted to implant has a relative mtDNA quantity greater than about 0.005.

In some embodiments, a quantity of mtDNA is determined by NGS and the threshold relative mtDNA quantity to determine implantation is 0.07. A preimplantation embryo having a relative mtDNA quantity less than about 0.07 is predicted to implant. For example, a preimplantation embryo predicted to implant can have a relative mtDNA quantity of about 0.068, about 0.065, about 0.0625, about 0.06, about 0.055, about 0.05, about 0.045, about 0.04, about 0.035. about 0.03, about 0.025, or about 0.02. In some embodiments, a preimplantation embryo predicted to implant has a relative mtDNA quantity less than 0.06. In some embodiments, a preimplantation embryo predicted to implant has a relative mtDNA quantity less than 0.05.

A preimplantation embryo having a relative mtDNA quantity greater than about 0.07 is unable to implant. For example, a preimplantation embryo predicted to not implant can have a relative mtDNA quantity of about 0.075, about 0.08, about 0.09, about 0.10, about 0.11, about 0.12, about 0.13, about 0.14, about 0.15, about 0.16, about 0.18, about 0.20, about 0.22, about 0.25, about 0.28, about 0.30, or about 0.32. In some embodiments, a preimplantation embryo predicted to implant has a relative mtDNA quantity greater than 0.08. In some embodiments, a preimplantation embryo predicted to implant has a relative mtDNA quantity greater than 0.10.

Additionally, the numerical value assigned to the threshold itself will differ depending on the technology used to examine the embryos (e.g. the threshold for quantification using Next-Gen Sequencing may have a different numerical value to that obtained from quantitative PCR).

### Amplification of a Target Sequence

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3rd ed. Cold Spring Harbor Laboratory Press: Cold Spring Harbor, N.Y.; Sambrook et al., (1989) Molecular Cloning: A Laboratory Manual. 2nd ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4th ed. Wiley-Liss; among others. The Current Protocols listed above are updated several times every year

Methods of amplifying a nucleic acid target sequence (e.g., a mtDNA or a reference DNA), are well known in the art and include, for example, polymerase chain reaction (PCR). In some embodiments, amplification of a target sequence includes real-time PCR. Real-time PCR can be standard real-time PCR or fast real-time PCR. It will be understood by a skilled person that in order to accommodate fast real-time PCR modifications may need to be made to, for example, instrumentation (e.g., to execute faster changes in temperature), enzymes (e.g., faster enzymes that maintain accuracy), and/or cycling parameters (e.g., shortening or even combining PCR steps).

Real-time PCR includes thermal cycling (i.e., cycles of repeated heating and cooling) of at least three steps: a first denaturing step to separate the two strands of the DNA double helix, a second annealing step which allows primers to bind to the DNA template, and a third extension step which facilitates DNA synthesis carried out by a DNA polymerase (e.g., a thermostable DNA polymerase). In some cases, a fourth step, and a moderately high temperature (e.g., 80° C.) can be included during which fluorescence can be measured.

The first step includes a high temperature which breaks the hydrogen bonds that hold double-stranded DNA together. The first step can include a temperature of about 95° C. The first step can include a time of about 10 seconds to 1 minute. For example, the first step can last about 15 seconds. In some embodiments, the first step includes a temperature of 95° C. and lasts 15 seconds.

The second step includes a lower temperature so that the PCR primers can bind to the DNA target sequence. The second step can include a temperature of about 50-60° C. For example, the temperature can be about 50° C., about 52° C., about 54° C., about 55° C., about 57° C., about 59° C., or about 60° C. The second step can can last about 15 seconds, about 30 seconds, or about 60 seconds. In some embodiments, the second step includes a temperature of 55° C. and lasts 15 seconds.

The third step includes an intermediate temperature which allows the DNA polymerase (e.g., a thermostable DNA polymerase) to extend the primer along the DNA target sequence. The third step can include a temperature of about 58-72° C. For example, the temperature can be about 58° C., about 62° C., about 65° C., about 68° C., about 70° C., or about 72° C. The third step can last about 45 seconds, about 60 seconds, or about 90 seconds. In some embodiments, the third step includes a temperature of 60° C. and lasts 1 minute.

These steps are typically repeated (i.e., cycled) 25-50 times. As will be understood by a person of skill in the art, there are three phases in of PCR amplification: 1) the exponential phase during which reagents are fresh and available and exact doubling of product occurs at every cycle (assuming 100% reaction efficiency); 2) the linear phase during which some of the reagents are consumed as a result of amplification and the PCR product is no longer doubled at each cycle; and 3) the plateau phase during which the reaction has stopped. In some embodiments, the number of cycles does not exceed the exponential phase. For example, the thermal cycling steps can be repeated 25 times, 30 times, 32 times, 35 times, 38 times, 40 times, 45 times, or 50 times. In some embodiments, the thermal cycle steps are repeated 35 times.

Real-time PCR thermal cycling can be preceded by an extended hold at a high temperature (e.g., about 95° C.) to activate a thermostable DNA polymerase which are inactive at room temperature. Cycle settings included such an extended hold are often referred to as “hot-start” cycles. The extended hold can be about 20 seconds to about 10 minutes. As will be understood by the skilled artisan, various thermostable DNA polymerases require different activation times. For example, some thermostable DNA polymerases (e.g., Taq DNA polymerase require a 10-minute activation at 95° C., where other thermostable DNA polymerases (e.g., AmpliTaq® Fast DNA polymerase) require only a 20-second activation at 95° C. In some embodiments, real-time PCR includes a 10 minute extended hold at about 95° C.

As will be understood by a person of skill in the art, thermal cycle parameters may need to be adjusted based on many factors. For example, the optimal primer annealing temperature may be dependent on the base composition (i.e., the proportion of A, T, and C nucleotides), primer concentration, and ionic reaction environment. For example, the extension time may be dependent on the amplicon length (i.e., extension typically requires about 1 minute/kb).

In some embodiments, real-time PCR includes a 10 minute extended hold at about 95° C. and thermal cycling including a first step at about 95° C. for about 15 seconds, a second step at about 50-60° C. for about 15 seconds, and a third step at about 68-72° C. for about 1 minute where the first, second, and third step are cycled about 35 times.

PCR includes at least two primers (i.e., a “primer pair” or a “primer set”) containing sequences complementary to a region of mtDNA sequence. A primer is a short synthetic oligonucleotide molecule which can be used to initiate the synthesis of a longer nucleic acid sequence. A primer can be annealed to a complementary target DNA sequence (e.g., mtDNA) by nucleic acid hybridization to form a hybrid between the primer and the target DNA sequence, and then the primer extended along the target DNA sequence by a DNA polymerase enzyme. A set of at least two primers (e.g., a forward primer and a reverse primer) which flank the target DNA sequence can be used to amplify the target DNA sequence to produce an amplification product (also referred to as an amplicon). A primer that can be used with the disclosed methods can be about 10-50 nucleotides, for example about 12-50 nucleotides, 15-40 nucleotides, 15-30 nucleotides, 12-40 nucleotides, 18 to 35 nucleotides, 18 to 30 nucleotides, 19 to 30 nucleotides, 19 to 29 nucleotides, or 20 to 29 nucleotides.

The target sequence can be one or more mtDNA sequences. PCR primers can be any primers designed to target any portion of mtDNA. The target mtDNA can be, for example, human mtDNA as set forth in NC 012920 (SEQ ID NO:1). A schematic showing the location of human mtDNA targets is set forth in FIG. 1. For example, PCR primers can target, without limitation, a mitochondrial DNA sequence encoding a 12S RNA, a mitochondrial DNA sequence encoding a 16S RNA, a mitochondrial DNA sequence encoding NADH dehydrogenase subunit 1 (MT-ND1), a mitochondrial DNA sequence NADH dehydrogenase subunit 1 (MT-ND1), a mitochondrial DNA sequence encoding NADH dehydrogenase subunit 2 (MT-ND2), a mitochondrial DNA sequence encoding NADH dehydrogenase subunit 3 (MT-ND3), a mitochondrial DNA sequence encoding NADH dehydrogenase subunit 4 (MT-ND4), a mitochondrial DNA sequence encoding NADH dehydrogenase subunit 5 (MT-ND5), a mitochondrial DNA sequence encoding Cytochrome b, mitochondrial Cytochrome c oxidase subunit 1, 2 or 3, a mitochondrial DNA sequence encoding an ATP synthase. Tools and strategies for designing PCR primers are known in the art.

In some embodiments, PCR primers can be designed to target the mitochondrial DNA sequence encoding 16S RNA (e.g., a human 16S sequence) as described in Fregel et al. (2011 Forensic Science International Genetics Supplement Series 3(1):e303-304). For example, a primer pair targeting a portion of human mtDNA that transcribes a 16S RNA can include a forward primer including the sequence GGTGATAGCTGGTTGTCCAAGAT (SEQ ID NO:2) and a reverse primer including the sequence

(SEQ ID NO: 3)
CCTACTATGGGTGTTAAATTTTTTACTCTCTC.

In some embodiments, PCR primers can be designed to target the mitochondrial DNA sequence encoding NADH-ubiquinone oxidoreductase chain 4 (MT-ND4) (e.g., a human MT-ND4 sequence). For example, a primer pair targeting a portion of human mtDNA that encodes MT-ND4 can include a forward primer including the sequence CTGTTCCCCAACCTTTTCCT (SEQ ID NO:8) and a reverse primer including the sequence

(SEQ ID NO: 9)
CCATGATTGTGAGGGGTAGG.

In some embodiments, PCR primers can be designed to target the mitochondrial DNA sequence encoding NADH dehydrogenase subunit 5 (NADH5) (e.g., a human NADH5 sequence) as described in Kavlick et al. (U.S. Pat. No. 9,080,205; issued Jul. 14, 2015).

The target sequence can be chromosomal DNA. Relative quantification is based on comparing the amount of the target DNA (e.g., mtDNA) in the sample to the amount of a reference DNA (e.g. chromosomal DNA) in the sample. The reference DNA can be any chromosomal DNA (e.g., a housekeeping gene, a multicopy gene or a repetitive sequence gene). For example, a reference DNA can be an Alu sequence, the L1 sequence, glyceraldehyde 3-phosphate dehydrogenase (GAPDH), or β-actin (ActB), Albumin, β-Globin or 18SrRNA. In some embodiments, a reference DNA can be a multicopy sequence in the human nuclear genome (e.g. Alu or L1). In some embodiments, the use of multiple individual DNA fragments as reference controls, summing their results together to produce a single reference value, is contemplated. However, virtually any combination of DNA sequences in the genome could be used for this purpose, so there is no point in attempting to specify them. Primers and/or probes directed to reference DNA sequences can be designed and/or synthesized. Alternatively, primers and/or probes directed to reference DNA sequences can be obtained commercially.

In some embodiments, PCR primers can be designed to target an Alu sequence (e.g., a human Alu sequence) as a reference DNA. For example, a primer pair targeting a human Alu sequence can include a forward primer including the sequence GTCAGGAGATCGAGACCATCCT (SEQ ID NO:5) and a reverse primer including the sequence AGTGGCGCAATCTCGGC (SEQ ID NO:6).

PCR amplification of mtDNA can be singleplex PCR (amplification of the target mtDNA and amplification of the reference DNA are performed in independent reactions; e.g., in separate tubes or wells) or duplex PCR (amplification of the target mtDNA and amplification of the reference DNA are performed in the same reaction; e.g., in the same tube or well).

Any of the PCR reagents described herein can be provided together as a reaction mixture. For example, a reaction mixture can include two or more of a primer pair, deoxynucleotides (dNTPs; with or without dUTP), a buffer (e.g., Tris-EDTA (TE) buffer), a DNA polymerase (e.g., a thermostable DNA polymerase such as a Taq DNA polymerase or an AmpliTaq® DNA polymerase), and/or nuclease free water. Commercially available reaction mixtures (e.g., TaqMan® Universal Master Mix) including deoxynucleotides, a buffer, and a DNA polymerase can be obtained and a primer pair can be added to the commercially available reaction mixture.

### Detection of an Amplified Target Sequence

Quantitative PCR (e.g., real-time PCR) includes detection of an amplicon. An amplicon that can be used with the disclosed methods can be about between 70-200 nucleotides, for example about 80-180 nucleotides, 90-170 nucleotides, 100-160 nucleotides, or 110-150 nucleotides. An amplicon can be detected by any suitable means (e.g., fluorescence). For example, the amplification of a target sequence can generate fluorescence, and detection of an amplicon can include detection of the fluorescence. The generation of fluorescence in real-time PCR can include, for example, a non-specific fluorescent dye that intercalate with any double-stranded DNA, or a sequence-specific probe consisting of oligonucleotides that are labelled with a fluorescent reporter dye which permits detection only after hybridization of the probe with its complementary sequence.

In cases where real-time PCR includes a non-specific fluorescent dye that intercalates with any double-stranded DNA, any DNA intercalating fluorescent dye can be used. Non-limiting examples of DNA intercalating fluorescent dyes include LCGreen® dyes, SYTO® dyes, EvaGreen® dyes, SYBR® Green dyes, SYBR® Gold dyes, Chromofy™ dyes, oxazole yellow, thiazole orange, and picogreen. As will be understood by a person of skill in the art, the presence of one of more fluorescent dyes can affect the melting temperature of DNA. As such, adjustment of the high temperature (e.g., by about 1-3° C.) of the first step of real-time PCR thermal cycling parameters can be increased or decreased accordingly.

In cases where real-time PCR includes a fluorescently labelled probe, any suitable probe can be used. A probe is an oligonucleotide that is complementary or substantially complementary to a region of an amplicon and can be used to detect or capture an amplicon. For example, a probe suitable for use in amplification-based detection methods can be designed from any sequence positioned within the amplicon. A probe that can be used with the disclosed methods can be about 10-50 nucleotides, for example about 12-50 nucleotides, 15-40 nucleotides, 15-30 nucleotides, 12-40 nucleotides, 18 to 35 nucleotides, 18 to 30 nucleotides, 19 to 30 nucleotides, 19 to 29 nucleotides, or 20 to 29 nucleotides.

In some embodiments, a probe can target an amplicon produced by a primer pair that targets a portion of mtDNA that transcribes a 16S RNA (e.g., a human mtDNA 16S). For example, a probe targeting a human mtDNA 16S amplicon can include the sequence

(SEQ ID NO: 4)
AATTTAACTGTTAGTCCAAAGAG.

In some embodiments, a probe can target an amplicon produced by a primer pair that targets a portion of mtDNA that encodes MT-ND4 (e.g., a human mtDNA encoding MT-ND4). For example, a probe targeting a human MT-ND4 amplicon can include the sequence

(SEQ ID NO: 10)
GACCCCCTAACAACCCCC.

In some embodiments, PCR primers can be designed to target the portion of mtDNA that encodes NADH dehydrogenase subunit 5 (NADH5) (e.g., a human NADH5 sequence).

In some embodiments, a probe can target an amplicon produced by a primer pair that targets a reference DNA (e.g., a human Alu sequence). For example, a probe targeting a human Alu amplicon can include the sequence AGCTACTCGGGAGGCTGAGGCAGGA (SEQ ID NO:7).

In cases where real-time PCR includes a fluorescently labelled probe, any suitable fluorescent reporter dye can be used. Non-limiting examples of fluorescent reporter dyes include NED™, cyanines (e.g., Cy™2, Cy™3, Cy™3.5, Cy™5, Cy™5.5, and Cy™7), fluoresceins (e.g., fluorescein isothiocyanate (FITC), and FAM phosphoramidite), rhodamines (e.g., carboxytetramethylrhodamine (TAMRA™), tetramethylrhodamine (TMR), tetramethylrhodamine (TRITC), sulforhodamine 101, Texas Red®, and Rhodamine Red®), and ROX™. The fluorescent reporter dye can be conjugated to either end of a probe to generate a fluorescently labelled probe. In cases where duplex PCR is used, it should be understood that a probe detecting the mtDNA amplicon and a probe detecting the reference DNA amplicon require different fluorescent reporter dyes.

In some cases, a fluorescently labelled probe includes a fluorescent reporter dye at one end and a quencher molecule at the opposite end. The close proximity of the quencher molecule to the fluorescent reporter dye allows the quencher molecule to eliminate or reduce emission (e.g., by absorbing the excitation energy) of the fluorescent reporter dye. The 5′ to 3′ exonuclease activity of a DNA polymerase breaks the physical reporter-quencher proximity and thus allows unquenched emission of the fluorescent reporter dye. Non-limiting examples, of quencher molecules include a nonfluorescent quencher (NFQ), dimethylaminoazobenzenesulfonic acid (DABSYL), Black Hole Quenchers, Qxl quenchers, Iowa black FQ, Iowa black RQ, IRDye QC-1. As will be understood by a person of skill in the art, quencher molecules are typically most effective at particular ranges of fluorescent emission. For example, DABSYL absorbs in the green spectrum and is often used with fluorescein.

A probe can include a groove binder (MGB) moiety. A MGB moiety increases the stability of the duplex formed when a probe anneals to an amplicon. The MGB moiety can be conjugated to one end of a probe. In cases where the probe is a fluorescently labelled probe, a MGB moiety can be located between the probe and a fluorescent label or a quencher or a MGB moiety can be located terminal to a fluorescent label or a quencher.

In some embodiments, a probe is a fluorescently labelled probe (e.g., including the sequence AATTTAACTGTTAGTCCAAAGAG (SEQ ID NO:4)) which includes a FAM fluorescent reporter dye at one end and a MBG moiety and a NFQ quencher molecule at the opposite end.

In some embodiments, a probe is a fluorescently labelled probe (e.g., including the sequence GACCCCCTAACAACCCCC (SEQ ID NO:10)) which includes a NED fluorescent reporter dye at one end and a NFQ quencher molecule at the opposite end.

In some embodiments, a probe is a fluorescently labelled probe (e.g., including the sequence AGCTACTCGGGAGGCTGAGGCAGGA (SEQ ID NO:7)) which includes a FAM fluorescent reporter dye at one end and a MBG moiety and a NFQ quencher molecule at the opposite end.

As demonstrated herein, mtDNA quantity can be used to determine implantation potential of a blastocyst. There is a threshold quantity of mtDNA above which implantation of a blastocyst (e.g., a euploid blastocyst) was never observed. This cut-off remained valid regardless of other considerations such as embryo morphology or the clinic where the patients were receiving treatment.

### Quantification of a Target Sequence

As used herein, “determining the quantity of DNA” refers to quantifying the amount of nucleic acid present in a sample.

In some methods herein, it is desirable to detect and quantify DNA present in a sample. Detection and quantification of DNA can be achieved by any one of a number of methods well known in the art. Using the known sequences for mtDNA or reference DNA sequences, specific probes and primers can be designed for use in the detection methods described below as appropriate.

Methods of quantifying a nucleic acid target sequence (e.g., mtDNA or chromosomal DNA), are well known in the art and include, for example, real-time polymerase chain reaction (PCR), next generation sequencing (NGS), spectrophotometric quantification, and UV fluorescence in presence of a DNA dye. The disclosure is not limited to particular quantification methods. In some embodiments, quantification of mtDNA includes real-time PCR.

Real-time PCR can be used quantitatively (quantitative real-time PCR), semi-quantitatively (semi quantitative real-time PCR) or qualitatively (qualitative real-time PCR). As will be understood in the field, PCR includes amplification of a single copy or a few copies of a piece of DNA across several orders of magnitude. In some embodiments, the real-time PCR is quantitative.

In relative quantification, the amount of the target sequence (e.g., mtDNA) in a sample is analyzed relative to the amount of a reference DNA (e.g., chromosomal DNA) in the same sample. As described in herein, a relative amount of mtDNA can be determined by many means including, but not limited to, real-time PCR or next generation sequencing (NGS).

In some embodiments, a relative amount of mtDNA is determined by real-time PCR. Methods using real-time PCR to quantify a target DNA include, for example, a comparative C_{T} (ΔC_{T}) method (relative quantitation), a relative standard curve method (relative quantitation), and a standard curve method (absolute quantitation).

The relative amount of mtDNA can be calculated via the equation 2^{-ΔΔCT} as described, for example, by Livik et al. (2001 METHODS 25:402-408). Real-Time PCR focuses on the exponential phase calculates a detection threshold (i.e., the level of detection at which a reaction reaches a fluorescent intensity above background) and a threshold cycle (C_{T}; i.e., the cycle number at which the sample crosses the detection threshold) for each sample.

The ΔC_{T} value describes the difference between the C_{T} value of the target mtDNA and the C_{T} value of the reference DNA (ΔC_{T}=Target DNA C_{T}−Reference DNA C_{T}) in a sample, and is used to normalize for the amount of template used within the sample. For example, ΔCt can be (mtDNA 16S C_{T})−(Alu C_{T}) or ΔC_{T} can be (MT-ND4 C_{T})−(Alu C_{T}). The ΔC_{T} value can be determined for multiple samples; e.g., a sample embryo, implanting embryos, and/or non-implanting embryos. In some embodiments, a ΔC_{T} value can be calculated using the mean C_{T} obtained from multiple (e.g., duplicate, triplicate, etc.) runs of a single sample.

The ΔΔC_{T} value describes the difference between the average ΔC_{T} value of the mtDNA in a first sample (e.g., a sample embryo) and the average ΔC_{T} value of the mtDNA in a second sample (e.g., an implanting embryo or a non-implanting embryo). For example, ΔΔC_{T} can be mtDNA^{implanting}ΔCT−mtDNA^{non-implanting}ΔCT.

The relative quantity of mtDNA in implanting embryos and the relative quantity of mtDNA in non-implanting embryos are used to determine threshold value which allows a clinician to determine the implantation potential of an embryo.

### Methods of Using

Methods provided herein include methods for determining an implantation threshold, methods for selecting an embryo for implantation, and methods for determining the implantation potential of an embryo (e.g., a euploid embryo).

Methods for determining an implantation threshold include, for example, providing a first DNA sample obtained from an implanting embryo and determining an amount of mitochondrial DNA (mtDNA) in the first DNA sample, providing a second DNA sample obtained from a non-implanting embryo and determining an amount of mtDNA in the second DNA sample. Methods for determining an implantation threshold also include comparing the amount of mtDNA in the first DNA sample to the amount of mtDNA in the second sample to determine an implantation potential threshold.

For example, the relative amount of mtDNA in a first DNA sample (e.g., a DNA sample from an implanting embryo) can be compared to the relative amount of mtDNA in a second DNA sample (e.g., a DNA sample from a non-implanting embryo). Analysis of the relative quantity of mtDNA in implanting embryos versus non-implanting embryos allows the establishment of a threshold for determining the potential of an embryo to implant (i.e., the “implantation potential”) and initiate a pregnancy.

Methods for determining the implantation potential of an embryo include, for example, providing a DNA sample obtained from an embryo, determining an amount of mitochondrial DNA (mtDNA) in the DNA sample, and determining an amount of a reference DNA in the DNA sample. Methods for determining the implantation potential of an embryo also include comparing the amount of mtDNA to the amount of reference DNA to determine a relative quantity of mtDNA in the embryo where the relative quantity of mtDNA in the embryo is indicative of the implantation potential of the embryo.

For example, the amount of mtDNA in a DNA sample (e.g., a DNA sample from a sample embryo) can be compared to the relative amount of reference DNA in the same DNA sample to establish a relative amount of mtDNA. Analysis of the relative quantity of mtDNA allows a clinician to determine the potential of an embryo to implant (i.e., the “implantation potential”) and initiate a pregnancy. In some cases, the relative amount of mtDNA in a DNA sample (e.g., a DNA sample from a sample embryo) can be compared to an established threshold for determining an implantation potential of an embryo. For example, a relative amount of mtDNA in a first DNA sample (e.g., a DNA sample from an implanting embryo) which falls below the threshold for determining an implantation potential of an embryo is indicative of a favorable implantation potential for the embryo (i.e., the embryo is likely to implant). For example, a relative amount of mtDNA in a first DNA sample (e.g., a DNA sample from an implanting embryo) which exceeds the threshold for determining an implantation potential of an embryo is indicative of an unfavorable implantation potential for the embryo (i.e., the embryo will not implant).

Methods provided herein can include providing a DNA sample obtained from an embryo (e.g., a sample embryo, an implanting embryo, or a non-implanting embryo). A sample embryo is an embryo with unknown implantation potential (e.g., an embryo prepared for in vitro fertilization). In some aspects, the methods described herein are applicant for research or clinical determinations, as well as in the generation of embryonic stem cell lines. A DNA sample obtained from an embryo can be DNA obtained from any embryonic source (e.g., tissue, cells, medium in which the embryo has been cultured, fluid from within the blastocoel cavity of embryos). A DNA sample obtained from an embryo contains both mtDNA and chromosomal DNA from the embryo. A DNA sample obtained from an embryo can be obtained from a preimplantation embryonic source. In some embodiments, a DNA sample obtained from a preimplantation embryo is obtained from the embryo 1, 2, 3, 4, 5, 6, or 7 days post fertilization or 1-2 days, 1-3 days, 3-5 days or 4-7 or 1-7 days post fertilization. Preimplantation embryonic sources of DNA include, for example, trophectoderm (TE), blastocyst, blastomere, medium in which the embryo has been cultured, fluid from within the blastocoel cavity of embryos. In some embodiments, a DNA sample is obtained from a TE sample. A DNA sample obtained from an embryo can be amplified (e.g., prior to performing quantification of the mtDNA). Methods amplifying a DNA sample include, for example, whole genome amplification methods including but not limited to multiple displacement amplification (MDA), multiple annealing and looping based amplification (MALBAC), methods based upon ligation of adapters followed by PCR (e.g. SurePlex, PicoPlex, GenomePlex), degenerate oligonucleotide primed PCR (DOP-PCR), multiplex PCR.

An embryo (and thus a DNA sample obtained from an embryo) can be any mammalian embryo. Non-limiting examples of mammals include, for example, humans, nonhuman primates (e.g. apes and monkeys), cattle, horses, sheep, rats, mice, pigs, and goats. In some embodiments, the sample can be obtained from a human embryo.

Methods provided herein can include determining an amount of mtDNA in a DNA sample obtained from an embryo as described herein. The amount of mtDNA in a DNA sample obtained from an embryo can be determined by quantitative PCR (e.g., real-time PCR). For example, determining the amount of mtDNA in a DNA sample obtained from an embryo can include determining the amount of mtDNA sequence that transcribes a 16S RNA using primers (e.g., SEQ ID NOs:2-3) and, optionally, probes (e.g., SEQ ID NO:4) as described herein. For example, determining the amount of mtDNA in a DNA sample obtained from an embryo can include determining the amount of mtDNA sequence that encodes MT-ND4 using primers (e.g., SEQ ID NOs:8-9) and, optionally, probes (e.g., SEQ ID NO:10) as described herein.

Methods provided herein can include determining an amount of a reference DNA in a DNA sample obtained from an embryo as described herein. The amount of reference DNA in a DNA sample obtained from an embryo can be determined by quantitative PCR (e.g., real-time PCR). For example, determining the amount of reference DNA in a DNA sample obtained from an embryo can include determining the amount of a sequence in the reference DNA using primers and optionally probes, e.g., determining the amount of an Alu sequence using primers (e.g., SEQ ID NOs:5-6) and, optionally, probes (e.g., SEQ ID NO:7) as described herein.

### Kits

Also provided herein are kits useful for performing the methods described herein. In some aspects, a kit provided herein includes a first synthetic oligonucleotide primer pair directed against a first mitochondrial DNA sequence and a second synthetic oligonucleotide primer pair directed against a reference chromosomal gene sequence DNA.

The first synthetic oligonucleotide primer pair can be directed against a mitochondrial DNA sequence as described herein (e.g., a mitochondrial DNA sequence encoding a 12S RNA, a mitochondrial DNA sequence encoding a 16S RNA, a mitochondrial DNA sequence encoding NADH dehydrogenase subunit 1 (MT-ND1), a mitochondrial DNA sequence NADH dehydrogenase subunit 1 (MT-ND1), a mitochondrial DNA sequence encoding NADH dehydrogenase subunit 2 (MT-ND2), a mitochondrial DNA sequence encoding NADH dehydrogenase subunit 3 (MT-ND3), a mitochondrial DNA sequence encoding NADH dehydrogenase subunit 4 (MT-ND4), a mitochondrial DNA sequence encoding NADH dehydrogenase subunit 5 (MT-ND5), a mitochondrial DNA sequence encoding Cytochrome b, mitochondrial Cytochrome c oxidase subunit 1, 2 or 3, a mitochondrial DNA sequence encoding an ATP synthase). In some embodiments, the kit includes a synthetic oligonucleotide primer pair directed against the mitochondrial DNA sequence encoding the 16S RNA gene sequence and, optionally, a probe directed against the mitochondrial DNA sequence encoding the 16S RNA. In some embodiments, the kit includes synthetic oligonucleotide primer pair directed against the mitochondrial DNA sequence encoding the NADH-ubiquinone oxidoreductase chain 4 (MT-ND4) and, optionally, a probe directed against the mitochondrial DNA sequence encoding the MT-ND4.

The reference synthetic oligonucleotide primer pair can be directed against the Alu sequence, the L1 sequence, glyceraldehyde 3-phosphate dehydrogenase (GAPDH), or β-actin (ActB), Albumin, β-Globin or 18SrRNA. In some embodiments, a reference DNA can be a multicopy sequence in the human nuclear genome (e.g. Alu or L1). In some embodiments, the kit includes a synthetic oligonucleotide primer pair directed against the Alu sequence and, optionally, a probe directed against the Alu sequence.

The kits of the invention can take on a variety of forms. Typically, a kit will include reagents suitable for determining the presence of or quantifying DNA in a sample. Optionally, the kits may contain one or more control samples. Also, the kits, in some cases, will include written information (indicia) providing a reference (e.g., predetermined values), wherein a comparison between the nucleic acid levels in the embryo and the reference (predetermined values) is indicative of a clinical status.

In some cases, the kits comprise software useful for comparing DNA levels or occurrences with a reference (e.g., a prediction model). Usually the software will be provided in a computer readable format such as a compact disc, but it also may be available for downloading via the internet. However, the kits are not so limited and other variations with will be apparent to one of ordinary skill in the art. The present methods can also be used for selecting a treatment and/or determining a treatment plan for a subject, based on the expression levels of a gene set (e.g., those disclosed herein).

Reference levels may be stored in a suitable data storage medium (e.g., a database) and are, thus, also available for future diagnoses. This also allows efficiently analysis because suitable reference results can be identified in the database once it has been confirmed (in the future) that the subject from which the corresponding reference sample was obtained did successfully implant. As used herein a “database” comprises data collected (e.g., analyte and/or reference level information and/or patient information) on a suitable storage medium. Moreover, the database, may further comprise a database management system. The database management system is, preferably, a network-based, hierarchical or object-oriented database management system. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative of mtDNA levels. Thus, if an identical or similar data set can be identified in the data collection, the test data set will be associated with implantation potential. Consequently, the information obtained from the data collection can be used to predict embryo implantation potential based on a test data set obtained from a reference embryo sample.

The invention further provides for the communication of assay results or diagnoses or both to technicians, physicians or patients, for example. In certain embodiments, computers will be used to communicate assay results or diagnoses or both to interested parties, e.g., physicians and their patients.

A kit provided herein can also include standard PCR reagents. PCR reagents are known to the skilled person and can include, for example, deoxynucleotides (dNTPs), a DNA polymerase (e.g., a thermostable DNA polymerase such as a Taq DNA polymerase or an AmpliTaq® DNA polymerase), a buffer such as a Tris-EDTA (TE) buffer, and/or nuclease free water.

A kit provided herein can also include a positive control and/or a negative control. In some cases the positive and/or negative controls can provide quality control for the amplification assay. For example, a positive control can be a DNA sample (e.g., a purified vector) that is known to have the sequence to be amplified and can be used to confirm that the primers and/or probe work properly; and a negative control can be a sample lacking any template for amplification (e.g., nuclease free water). In some cases, the positive and/or negative controls can provide DNA sequences with known quantitative and/or qualitative information. For example, a positive control can be a DNA sample obtained from an embryo known to implant; and a negative control can be a DNA sample obtained from an embryo known to not implant.

One or more of the reagents in a kit provided herein can be provided in a form that allows for ease in packaging and transport. For example, a buffer can be provided in a concentrated form (e.g., a 10× buffer), primer and/or probes can be provided in lyophilized form, etc.

The kit can include any appropriate packaging. For example, reagents that require refrigeration may be packaged with an ice pack. For example, reagents that require freezing may be packaged with dry ice.

The kit can include an instruction manual.

The invention will be further described in the following examples, which do not limit the scope of the invention described in the claims.

### Examples

The following examples investigate the biological and clinical relevance of the quantity of mtDNA in 379 embryos. The embryos were examined via a combination of microarray comparative genomic hybridisation (aCGH), quantitative PCR and next generation sequencing (NGS), providing information on chromosomal status, amount of mtDNA, and presence of mutations in the mitochondrial genome.

### Materials and Methods

### Ethics Statement

Ethical approval was obtained from Western IRB (20060680 and 20131473) and the NHS Health Research Authority (NRES Committee South Central). Two types of embryonic samples were assessed: blastomeres derived from cleavage stage embryos (3 days post-fertilization of the oocyte) and TE cells from blastocysts (5-6 days post-fertilization). All of the embryos included in this study underwent biopsy at the request of the couples who generated them, in order to analyze their chromosomes in the context of either preimplantation genetic screening (PGS) or preimplantation genetic diagnosis (PGD). The standard process used in our laboratory for this purpose involves whole genome amplification (WGA) followed by aCGH. The research described in this paper involved analysis of surplus amplified DNA only, leftover after PGD or PGS had been completed. The embryos were not subjected to any additional interventions and the clinical treatment of the patients was not altered as a result of this study. Informed patient consent for analysis of discarded amplified DNA was obtained under an approved protocol (see above).

### Patients and Samples

Surplus WGA samples were derived from TE biopsies (typically consisting of 5-10 cells) from a total of 340 blastocysts. The blastocysts were produced by 161 couples of an average female age of 38 years (range 26-42 years). The excess WGA products from single blastomeres were obtained from a total of 39 cleavage stage embryos. These embryos were generated by 32 couples. The average female age of this patient group was 37.4 years (age range 29-42 years). IVF clinics located in the USA and the UK participated in this investigation.

### Embryo Sampling and Cytogenetic Analysis Preparation

Embryo micromanipulation, biopsy, and preparation of biopsied material for chromosome analysis were as described previously (Fragouli et al., 2013 Hum Genet 132:1001-1013; Fragouli et al., 2011 Hum Reprod 26:480-490. All samples were analyzed with the use of a single, highly validated platform for microarray comparative genomic hybridisation (aCGH) (Fragouli et al., 2011 Hum Reprod 26:480-490; Wells et al., 2014 J Med Genet 51:553-562; Magli et al., 2011 Hum Reprod 26:3181-3185; Gutierrez-Mateo et al., 2011 Fertil Steril 95:953-958; Christopikou et al., 2013 Hum Reprod 28:1426-1434; Mertzanidou et al., 2013 Hum Reprod 28:1716-1724).

### Microarray CGH

Chromosome analysis was carried out using 24Sure Cytochip V3 microarrays (Illumina Ltd., Cambridge, UK). The protocol used was as described in Fragouli et al. (2013 Hum Genet 132:1001-1013). In brief, the procedure involved cell lysis and WGA (SurePlex, Rubicon Genomics, Ann Arbor, USA). This was followed by fluorescent labelling of amplified DNA samples, and two ‘reference’ DNAs (46,XY an 46,XX), and hybridization to the microarray. The microarrays were washed, scanned (InnoScan 710, Innopsys, Carbonne, France), and the resulting images analyzed using Blue-Fuse software (Illumina, Cambridge, UK). Using this approach, it was possible to determine the chromosome constitution of the blastomere or TE samples, allowing classification of the corresponding embryos as normal or aneuploid.

Relative Quantification of mtDNA Copy Number mtDNA copy number quantification took place initially via fluorescent real-time PCR assessment of embryonic samples. These had previously undergone WGA (SurePlex, Rubicon, USA), as part of the cytogenetic analysis described above. A custom-designed TaqMan Assay (AATTTAACTGTTAGTCCAAAGAG (SEQ ID NO:4); Life Technologies, UK) was used to target and amplify a specific mtDNA fragment (the mitochondrial 16s ribosomal RNA sequence (Fregel et al., 2011 Forensic Science International: Genetics Supplement Series 3:e303-e304). Normalisation of input DNA took place with the use of an additional TaqMan Assay targeting the multicopy Alu sequence (YB8-ALU-S68) (AGCTACTCGGGAGGCTGAGGCAGGA (SEQ ID NO:7); Life Technologies, UK). The purpose of normalization relative to a nuclear DNA sequence was to ensure that any variation in mtDNA levels related to technical issues (e.g. differences in the efficiency of WGA or the number of cells within the biopsy specimen) could be adjusted for. A multicopy sequence (i.e. Alu) was chosen for this purpose since, at the single cell level, single copy sequences may give spurious results due to factors such as allele drop-out (ADO). Each real-time PCR experiment included analysis of a reference DNA against which all samples were compared. The reference DNA was derived from a karyotypically normal male (46,XY) blastomere or TE sample, amplified via the SurePlex method (Rubicon, USA), and remained constant throughout the course of this study. A negative control (nuclease free H₂O and PCR master-mix) was also included for both sets of amplifications. Triplicate amplification reactions were set up for both the mtDNA and Alu sequences. Each reaction contained 1 μl of whole genome amplified (SurePlex) embryonic DNA, 8 μl of nuclease-free H2O, 10 μl of Taq-Man Universal Master-mix II (2×)/no UNG (Life Technologies, UK) and 1 μl of the 20× Taq-Man mtDNA or Alu assay (Life Technologies, UK), for a total volume of 20 μl. The thermal cycler used was a StepOne Real-Time PCR System (Life Technologies, UK), and the following conditions were employed: incubation at 50° C. for 2 min, incubation at 95° C. for 10 min and then 30 cycles of 95° C. for 15 s and 60° C. for 1 min.

### Mitochondrial Genome Analysis Via Next Generation Sequencing

A group of 23 WGA products from euploid TE samples with varying levels of mtDNA (previously established via real-time PCR) underwent massively parallel DNA sequencing using a MiSeq and a HiSeq (Illumina, USA). The protocol was as suggested by the manufacturer (Illumina, USA). Library preparation involved the initial purification of SurePlex amplified products with the use of the Zymo DNA Clean & Concentrator (Zymo Research Corporation, Irvine, Calif., USA), followed by quantification of DNA concentrations via the Qubit dsDNA HS Assay Kit (Life Technologies, USA). One nanogram of DNA was subsequently converted into dual-indexed sequencing libraries using the Nextera XT DNA Sample Preparation and Index Kits according to the manufacturer's protocol (Illumina, USA).

The libraries were sequenced 2×150 cycles with dual indexing on an Illumina MiSeq using the MiSeq Reagent Kit v3 or 2×100 cycles with dual indexing on an Illumina HiSeq 2000 using the TruSeq PE Cluster Kit v3-cBot-HS and TruSeq SBS kit v3-HS for flow cell clustering and sequencing respectively (Illumina, USA).

Reads were aligned to the human genome hg19 using bwa (Li et al., 2009 Bioinformatics 25:1754-1760) or iSAAC (Raczy et al., 2013 Bioinformatics 29:2041-2043) for MiSeq and HiSeq sequencing runs respectively. After alignment, unmapped reads, duplicate reads, reads with low mapping scores and reads with greater than one mismatch with the reference genome were removed using BEDtools (Quinlan et al., 2010 Bioinformatics 26:841-842) and SAMtools (Li et al., 2009 Bioinformatics 25:2078-2079). The reference genome was divided into non-overlapping bins such that each bin contains 100 uniquely mapping 36mers across the genome (Baslan et al., 2012 Nat Protoc 7: 1024-1041) and the number of reads that mapped to each bin was counted. The bin read count was normalized based on GC content and an in-silico reference data set in order to remove bias. The copy number per bin was calculated according to the formula:

$$Copy Number = \frac{Bin Read Count}{Median Bin Read Count} X 2$$

where the median autosomal read count is expected to correspond to copy number two. A 13-bin sliding median was used to smooth bin-wise copy number values for each chromosome. Copy-number status for each chromosome was derived from the median of smoothed copy-number values across the chromosome.

After alignment, genomeCoverageBed files were generated using BEDTools and the fraction of total sequenced bases that aligned to the mitochondrial genome relative to the nuclear genome was calculated. Using SAMtools, the mitochondrial reads were extracted from the BAM files and analyzed with the online tool MitoBamAnnotator (Zhidkov et al., 2011 Mitochondrion 11:924-928).

mtDNA Quantification Via NGS

An additional 38 TE samples underwent NGS analysis in order for chromosome constitution assessment and mtDNA quantification to take place. A different type of NGS technology was used for the analysis of these 38 TE samples, involving the application of the PGM (Life Technologies, UK). For this purpose a different initial WGA approach involving the use of multiple displacement amplification (MDA) was employed. Briefly, all 38 TE samples were lysed by adding 2.5 μl of alkaline lysis buffer (0.75 μl PCR-grade water [Promega, USA]; 1.25 μl DTT [0.1M] [Sigma, UK]; 0.5 μl NaOH [1.0M][Sigma, UK]), and then incubating them for 10 min at 65° C. in a thermal-cycler. MDA whole genome amplification took place with the use of the Repli-g Midi Reaction kit (Qiagen, UK) as was suggested by the manufacturer. All samples were incubated in a thermal-cycler at 30° C. for 120 minutes and 65° C. for a further 5 minutes. The NGS procedure used is described, for example, in Wells et al. (2014 J Med Genet 51:553-562).

### Statistical Analysis

The relative amount of mtDNA in relation to the Alu sequence for both reference and test samples was determined by the equation 2-Delta Delta Ct. The Delta Ct for reference and test samples was the end result of a data normalisation process. This involved the calculation of the Delta Ct for reference and test loci (Ct-mtDNA minus Ct-Alu), and the adjustment of the test samples values in relation to the reference DNA sample (Delta Ct plus Normalisation factor) (Schmittgen et al., 2008 Nat Protoc 3:1101-1108). Statistical analysis of the final mtDNA values utilised unpaired two-tailed t-tests. Parameters which were compared during this study included female age (younger vs. older), embryo chromosome status (normal vs. aneuploid), and embryo viability/implantation potential (ongoing pregnancy vs. failure to implant).

Relative quantification of mtDNA using NGS involved determination of the number of DNA sequence reads attributable to the mitochondrial genome as a fraction of the total number of reads. The great majority of DNA fragments sequenced are derived from the nuclear genome and provide a control for the number of cells in the biopsy specimen.

### Example 1: Cytogenetic Analysis of Embryonic Samples

A total of 39 cleavage stage embryos and 340 blastocysts, which had been cytogenetically tested, were studied during the course of this investigation. All of the cleavage stage embryos had been characterised as being chromosomally normal after microarray comparative genomic hybridization (aCGH) analysis and transferred to the uterus. Of the blastocysts examined, 302 were analysed using aCGH, and 38 using next generation sequencing (NGS) methodology. Of these, 123 were determined to be aneuploid (99 via aCGH analysis and 24 via NGS analysis), while the remaining 217 were characterised as being chromosomally normal (203 via aCGH analysis and 14 via NGS analysis). One hundred and thirty one of the normal blastocysts and all 39 euploid cleavage stage embryos underwent uterine transfer. Embryo classification as chromosomally normal or aneuploid was based on results obtained after aCGH or NGS analysis of either a single blastomere (cleavage stage), or 5-10 TE cells (blastocysts).

### Example 2: The Effect of Female Age on mtDNA Quantity

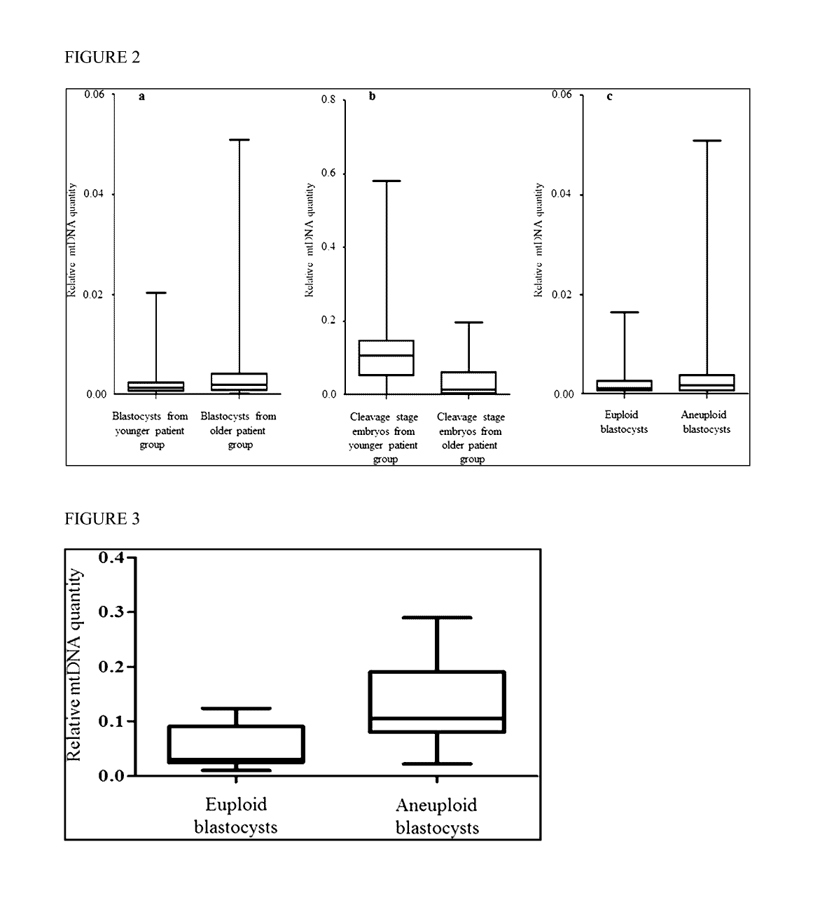

The relative amount of mtDNA was assessed in relation to female age. Specifically, an initial comparison of 148 blastocysts generated by a reproductively younger group of women (average age 34.8 years, range 26-37 years) and the 154 blastocysts generated by a reproductively older group (average age 39.8 years, range 38-42 years) was undertaken with the use of real-time PCR. Data analysis clearly showed a statistically significant increase (P=0.003) in the amount of mtDNA in blastocysts from the reproductively older women. This phenomenon was evident when all blastocysts were considered together, but was also apparent if chromosomally normal and abnormal embryos were considered separately (P=0.018 and P=0.05, respectively). The relative amounts of mtDNA in chromosomally normal and abnormal blastocysts for the female age groups under investigation are summarized in Table 1 and illustrated in FIG. 2a.

TABLE 1
The average relative quantities of mtDNA observed in association
to female age and blastocyst chromosome status.

| Female | Number of | | mtDNA quantity | Average mtDNA | mtDNA quantity | Average mtDNA |
|---|---|---|---|---|---|---|
| age | embryos | | range/euploid | quantity/euploid | range/aneuploid | quantity/aneuploid |
| (years) | assessed | | blastocysts | blastocysts | blastocysts | blastocysts |
| 26 | 1 | euploid | 0.002273 | N/A | N/A | N/A |
| 29 | 1 | euploid | 0.0029 | N/A | N/A | N/A |
| 30 | 9 | euploid | 0.000065-0.0017 | 0.000482 | 0.000095-0.0069 | 0.00161 |
| | 5 | aneuploid | | | | |
| 31 | 9 | euploid | 0.000093-0.00231 | 0.000998 | 0.00166-0.0044 | 0.00248 |
| | 3 | aneuploid | | | | |
| 32 | 7 | euploid | 0.000318-0.00462 | 0.00137 | 0.000324-0.00275 | 0.00145 |
| | 3 | aneuploid | | | | |
| 33 | 9 | euploid | 0.00013-0.002532 | 0.00115 | 0.00048-0.00218 | 0.00159 |
| | 3 | aneuploid | | | | |
| 34 | 8 | euploid | 0.001-0.0034 | 0.0043 | 0.00244 | 0.00244 |
| | 1 | aneuploid | | | | |
| 35 | 10 | euploid | 0.00076-0.00765 | 0.00265 | 0.00289-0.0034 | 0.00315 |
| | 2 | aneuploid | | | | |
| 36 | 26 | euploid | 0.00032-0.00968 | 0.002 | 0.00026-0.0082 | 0.00212 |
| | 9 | aneuploid | | | | |
| 37 | 29 | euploid | 0.000038-0.015 | 0.0023 | 0.000097-0.00416 | 0.00142 |
| | 12 | aneuploid | | | | |
| 38 | 28 | euploid | 0.000294-0.01 | 0.00283 | 0.00037-0.007 | 0.00285 |
| | 6 | aneuploid | | | | |
| 39 | 18 | euploid | 0.00063-0.0164 | 0.0025 | 0.00053-0.00722 | 0.00393 |
| | 18 | aneuploid | | | | |
| 40 | 19 | euploid | 0.0004-0.0064 | 0.002 | 0.00036-0.0038 | 0.002 |
| | 7 | aneuploid | | | | |
| 41 | 16 | euploid | 0.00025-0.006 | 0.0026 | 0.00027-0.05 | 0.0079 |
| | 15 | aneuploid | | | | |
| 42 | 13 | euploid | 0.00013-0.012 | 0.0048 | 0.0001-0.016 | 0.0053 |
| | 15 | aneuploid | | | | |

The mtDNA values (2-^{Delta Delta Ct}) were obtained during real-time PCR analysis.

A significant difference (P=0.01) in the levels of mtDNA according to female age was also observed at the cleavage stage. However, unlike the blastocyst stage, blastomeres removed from embryos generated by reproductively younger women (average age 33.7 years, range 29-37 years) were seen to contain higher mtDNA amounts, compared to those removed from embryos generated by reproductively older women (average age 39.2 years, range 38-42 years). These results are illustrated in FIG. 2b and Table 2.

TABLE 2
The average relative quantities of mtDNA observed in
association with female age at the cleavage stage.

| Female | Number of | | |
|---|---|---|---|
| age | embryos | mtDNA quantity | Average mtDNA |
| (years) | assessed | range | quantity |
| 29 | 1 | 0.1077 | N/A |
| 30 | 3 | 0.0673-0.5823 | 0.2912 |
| 33 | 4 | 0.1058-0.1716 | 0.139 |
| 34 | 3 | 0.00055-0.01389 | 0.0055 |
| 35 | 2 | 0.06480-0.1476 | 0.106 |
| 36 | 4 | 0.0504-0.1276 | 0.0831 |
| 37 | 2 | 0.00051-0.1176 | 0.059 |
| 38 | 9 | 0.00251-0.1967 | 0.0591 |
| 39 | 4 | 0.000297-0.00482 | 0.002 |
| 40 | 4 | 0.00927-0.0636 | 0.0254 |
| 42 | 3 | 0.00082-0.087 | 0.03 |

The mtDNA values (2-^{Delta Delta Ct}) were obtained during real-time PCR analysis. All examined blastomeres were characterised as being chromosomally normal.

### Example 3: The Relationship Between Embryo Chromosome Constitution and mtDNA Quantity

Chromosome abnormalities are extremely common during the earliest stages of embryo development, with rates decreasing post-implantation (Fragouli et al., 2013 Hum Genet 132:1001-1013). Real-time PCR assessment of mtDNA quantity in relation to chromosome status took place for a total of 203 normal and 99 aneuploid blastocyst stage embryos. TE samples from all these embryos were assessed with the use of aCGH. It was evident that chromosomally abnormal blastocysts tended to contain significantly larger amounts of mtDNA compared to those which were characterised as being euploid (P=0.025) (FIG. 2c).

To verify these results using an unrelated methodology, the inventors applied a different type of whole genome amplification (WGA) method followed by NGS to TE biopsies derived from 38 additional blastocysts. The advantage of NGS technology is its capability to simultaneously examine nuclear and mitochondrial genomes. NGS analysis demonstrated that 14 of the blastocysts were euploid, whereas chromosome abnormalities were scored for the remaining 24. This finding was confirmed via aCGH conducted using separate aliquots of each WGA product. As with the real-time PCR results, statistical analysis of NGS data showed a significant increase (P=0.006) in the quantity of mtDNA in aneuploid blastocysts compared to those that were chromosomally normal. This provided independent confirmation of the real-time PCR findings. The NGS mtDNA data are illustrated in FIG. 3.

It should be noted that although mtDNA quantity increases with advancing female age, the relationship with aneuploidy appears to be an independent factor. Within any given age group mtDNA levels were higher, on average, for blastocysts that were chromosomally abnormal (Table 1).

### Example 4: mtDNA Copy Number and the Ability of Blastocysts to Establish a Clinical Pregnancy

In order to assess whether mtDNA content had an influence on the ability of an embryo to implant and initiate a pregnancy, the inventors retrospectively analyzed data obtained from single embryo transfers (SETs) with or without implantation, or double embryo transfers (DETs) which either led to dizygotic twins or no implantation. Specifically the inventors examined the mtDNA content of 89 blastocysts, 81 of which were transferred in SETs with the remaining 8 being transferred in DETs. Eighty-five patients were included in this part of the study and the average female age was 38.3 years. Of the blastocysts transferred to these patients, 42 established an ongoing clinical pregnancy, while the remaining 47 failed to implant.

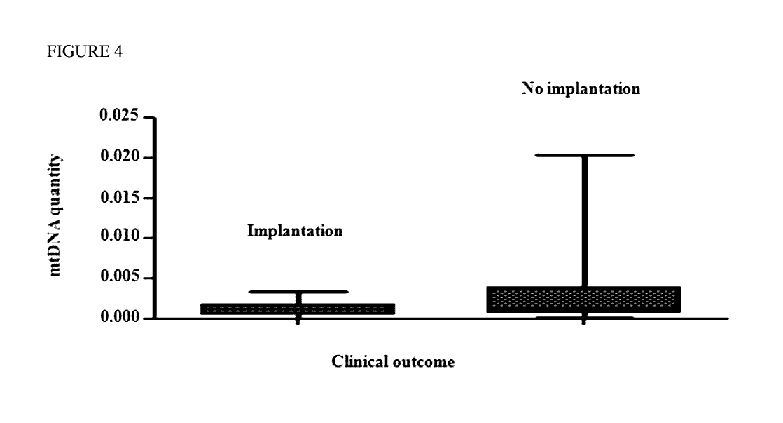

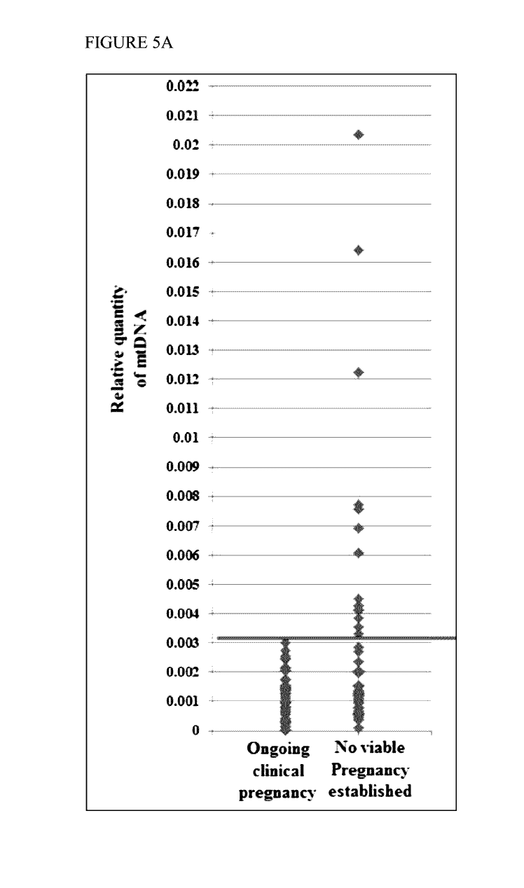

Real-time PCR analysis clearly showed that blastocysts able to implant contained significantly lower amounts of mtDNA compared to those incapable of initiating a clinical pregnancy (P=0.007). These results are summarized in FIG. 4.

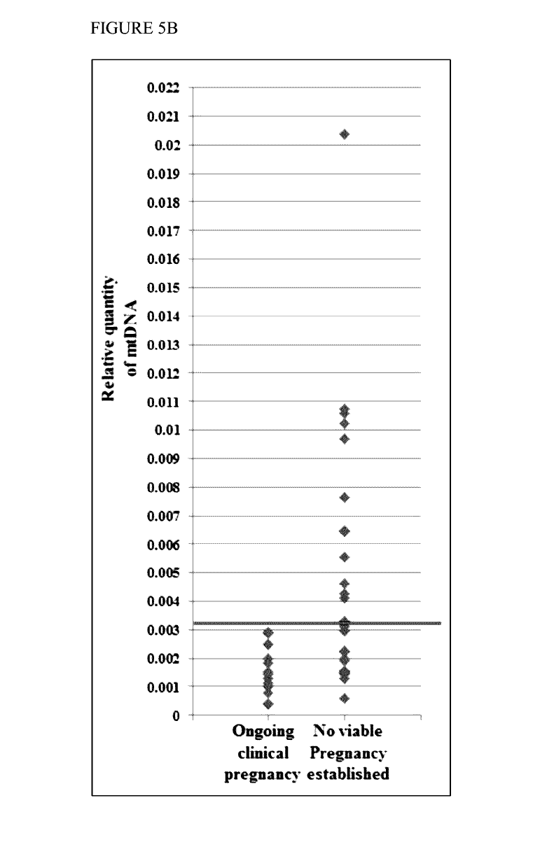

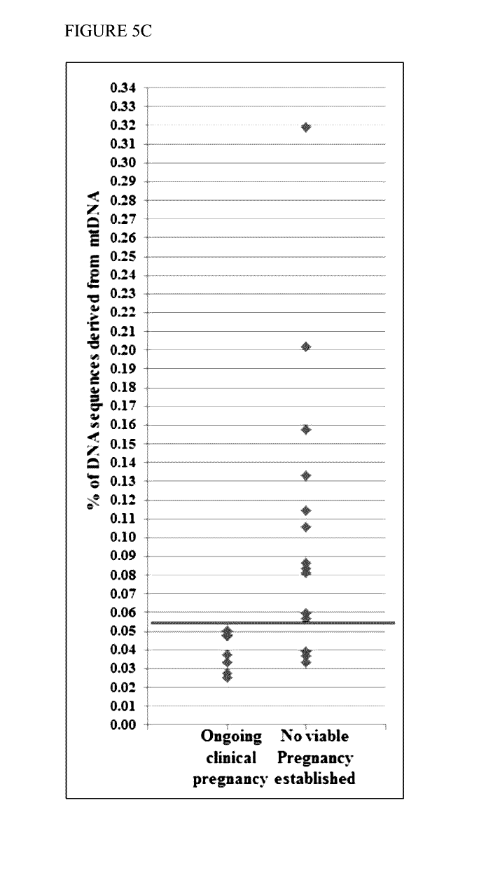

Analysis of the real-time PCR data obtained from implanting and non-implanting blastocysts allowed the establishment of an mtDNA quantity threshold above which implantation was never seen to occur. Specifically, 42/42 (100%) blastocysts which led to a clinical pregnancy contained relative mtDNA quantities lower than 0.003. Additionally, 14/14 (100%) of embryos with mtDNA quantities higher than 0.003 were unable to implant. These represented 30% (14 of 47) of the non-implanting blastocysts, while the remaining 70% (33 of 47) contained amounts of mtDNA below the threshold (FIG. 5A). It is of note that the identified mtDNA quantity implantation threshold of 0.003 was independent of blastocyst morphology, age and the IVF clinic that produced the embryos.

To further evaluate the association between elevated mtDNA levels and implantation failure, we analyzed 23 TE samples with the use of NGS. All of the embryos were euploid, and had previously been analyzed via real-time PCR. The clinical outcome after transfer was known for 21 of the corresponding blastocysts. Seven of these led to pregnancies whereas the remaining 14 failed to implant. Of the 14 embryos which had not implanted, real-time PCR identified 9 containing mtDNA amounts higher than 0.003. NGS analysis confirmed the real-time PCR findings, clearly demonstrating increased quantities of mtDNA in non-implanting embryos compared to those shown to be viable. Elevated mtDNA quantities were also observed for an additional 3 of the TE samples for which clinical outcome was not known. These results are illustrated in FIG. 5C and Table 3.

TABLE 3
mtDNA quantities and clinical outcomes of 23 TE samples assessed via real-time PCR and NGS.

| | mtDNA quantity/ | MtDNA quantity/ | mtDNA quantity/ | MtDNA quantity/ | |
|---|---|---|---|---|---|
| TE | Real-time PCR | Low (normal)/ | NGS (% of total | Low (normal)/ | Clinical outcome |
| sample | (2-^{Delta Delta Ct}) | High (abnormal)* | reads) | High (abnormal)* | after ET |
| A1 | 0.0004047 | Low/Normal | 0.05 | Low/Normal | Implantation |
| A2 | 0.000131 | Low/Normal | 0.03 | Low/Normal | Implantation |
| A3 | 0.001257 | Low/Normal | 0.05 | Low/Normal | Implantation |
| A4 | 0.00103 | Low/Normal | 0.04 | Low/Normal | No Implantation |
| A5 | 0.00026 | Low/Normal | 0.03 | Low/Normal | Implantation |
| A6 | 0.000406 | Low/Normal | 0.03 | Low/Normal | Implantation |
| A7 | 0.000669 | Low/Normal | 0.03 | Low/Normal | No Implantation |
| A8 | 0.00151 | Low/Normal | 0.04 | Low/Normal | Implantation |
| A9 | 0.00217 | Low/Normal | 0.05 | Low/Normal | Implantation |
| A10 | 0.001208 | Low/Normal | 0.06 | Low/Normal | No Implantation |
| A11 | 0.000548 | Low/Normal | 0.04 | Low/Normal | No Implantation |
| A12 | 0.001932 | Low/Normal | 0.04 | Low/Normal | Not known |
| B1 | 0.00341 | High/abnormal | 0.08 | High/abnormal | No Implantation |
| B2 | 0.0164158 | High/abnormal | 0.32 | High/abnormal | No Implantation |
| B3 | 0.00643 | High/abnormal | 0.20 | High/abnormal | No Implantation |
| B4 | 0.000602 | Low/Normal | 0.04 | Low/Normal | Not known |
| B5 | 0.01075 | High/abnormal | 0.11 | High/abnormal | No Implantation |
| B6 | 0.00222 | Low/Normal | 0.06 | Low/Normal | No Implantation |
| B7 | 0.0033 | High/abnormal | 0.11 | High/abnormal | No Implantation |
| B8 | 0.00426 | High/abnormal | 0.13 | High/abnormal | No Implantation |
| B9 | 0.00412 | High/abnormal | 0.16 | High/abnormal | No Implantation |
| B10 | 0.0060 | High/abnormal | 0.09 | High/abnormal | No Implantation |
| B11 | 0.0069 | High/abnormal | 0.08 | High/abnormal | No Implantation |

*The threshold for considering a sample to have elevated mtDNA levels, incompatible with implantation, was 0.003 for real-time PCR and 0.07 for NGS.

### Example 5: Blinded Prospective Prediction of IVF Outcome Based Upon mtDNA Quantification

Following establishment of a viability threshold for mtDNA levels in blastocysts, based upon retrospective data analysis, the inventors carried out a blinded prospective study to assess its predictive value. Quantification of mtDNA was carried out in TE biopsies from a total of 42 euploid blastocysts that had been selected for transfer to the uterus after chromosomal (aCGH) and morphological analyses. The average age of the women generating these embryos was 36.7 years (age range 26-42 years) and the couples were being treated in 6 different IVF clinics. Fifteen embryos were shown to have mtDNA levels above the 0.003 threshold and were therefore predicted to be associated with failure to establish a viable pregnancy (FIG. 4b). Review of biochemical and ultrasound data a few weeks later, confirmed that none of these embryo transfers had resulted in a viable pregnancy. Thus the negative predictive value of the mtDNA analysis was 100%. The remaining 27 embryos had mtDNA quantities below 0.003 and were therefore predicted to have some potential for producing a child. After decoding of the blinded results, it was found that 16 of these embryos had ultimately established viable clinical pregnancies. Therefore, 59% of the embryos classified by mtDNA analysis as potentially viable created an ongoing clinical pregnancy. This contrasts to the 38% (16/42) pregnancy rate achieved for this cohort of embryos, transferred without reference to the mtDNA results. These results further confirmed our previous findings that embryos with high mtDNA quantities are incapable of forming a clinical pregnancy. Moreover, it was demonstrated that mtDNA quantification can be used as an effective biomarker to assist selection among euploid embryos.

### Example 6: The Origin of Elevated Levels of mtDNA in Non-Implanting Embryos

In an attempt to shed light on whether the origin of excess mtDNA seen in non-implanting blastocysts was embryonic or was derived from the oocyte, the inventors examined mtDNA quantities in blastomeres removed from 39 cleavage stage embryos. Mitochondrial DNA replication is not thought to occur until the blastocyst stage, so the levels of mtDNA detected at earlier developmental stages are expected to reflect those in the oocyte. All of the cleavage stage embryos considered in this part of the study had been characterized as euploid following blastomere aCGH analysis and had been transferred to the uterus. As far as the clinical outcome was concerned, 17 embryos were capable of implanting, leading to clinical pregnancies, while the remaining 22 did not implant.

It was evident that blastomeres contained much higher levels of mtDNA, compared to TE samples. This was not an unexpected finding, considering the much larger cytoplasmic volume of blastomeres in comparison to TE biopsies.

Assessment of the data obtained during this analysis showed that there was no significant difference in the quantities of mtDNA in blastomeres derived from embryos which implanted compared with those from embryos that failed to implant (P=0.7). Therefore it was concluded that the increased mtDNA content seen in cells from a subset of non-viable blastocysts must originate after the cleavage stage. This conclusion is compatible with the notion that the first significant wave of mitochondrial genome replication begins after differentiation of embryonic cells into TE and inner cell mass is initiated.

### Example 7: Mitochondrial Genome Analysis

One potential reason for altered mtDNA levels could be a proliferation of mitochondria as a compensatory response to the presence of defective organelles harbouring mutations in key genes. To explore this possibility NGS was used to sequence the entire mitochondrial genome of 23 TE samples. The samples were derived from chromosomally normal blastocysts, 9 of which had elevated quantities of mtDNA (initially determined using real-time PCR) and 14 that had mtDNA levels in the normal range (Table 3). The mitochondrial genome was sequenced to an average depth of ˜150 reads, permitting mutation detection and an estimate of degree of heteroplasmy. Mutations, usually in heteroplasmic form, were seen to some extent in all samples, but were no more prevalent in blastocysts with high mtDNA levels than they were in embryos with lower quantities of mtDNA.

### DISCUSSION

Previous studies examining human mitochondria and mtDNA in relation to female reproductive aging have focused on the analysis of oocytes rather than embryos. Published results have not been entirely concordant, but most report that mtDNA levels either remain unchanged or decrease with advancing age [16, 21-22]. A reduction in the number of oocyte mitochondria with age has also been reported in older mice [23]. Other research has indicated that a decline in oocyte mtDNA copy number may be associated with ovarian pathology [1, 24]. During the current study a significant (P=0.01) decline in mtDNA quantities was observed in cells from cleavage stage embryos generated by reproductively older women, compared to those from younger patients. Considering that the main wave of mtDNA replication is thought to start after blastocyst formation [3, 8], these observations at an early preimplantation stage are likely to be representative of the quantities of mtDNA that were present in the corresponding oocytes. Our data are therefore supportive of the notion that oocyte mtDNA levels decrease with advancing female age.

Interestingly, analysis of specimens from human blastocysts, just two days after the cleavage stage, revealed a trend in the opposite direction, with mtDNA levels increasing significantly with advancing female age. This association was apparent for both euploid and aneuploid blastocysts. It is likely that the elevated quantities of mtDNA observed are indicative of an increase in the number of mitochondria, although the relationship between the two factors is complicated by the fact that a single organelle may contain more than one copy of the mitochondrial genome.

It is well established that the likelihood of an oocyte producing a viable embryo is inversely correlated with the age of the mother. This is clearly demonstrated by the significant difference in the success rate of IVF treatment for older patients using their own oocytes, compared to patients utilizing gametes donated by younger women. The increase in mtDNA with age seen at the blastocyst stage during the current study raises the question of whether mitochondria might play a direct role in the decline of female fertility with age.

It is conceivable that elevated mtDNA levels are a consequence of a compensatory mechanism, aimed at normalization of ATP generation in the face of growing numbers of compromised organelles of reduced function. Indeed, data obtained from animal models suggest a decline in the integrity of ‘older’ mitochondria and a consequent deterioration in the efficiency of ATP production [24, 25]. Mitochondria in the oocytes of older hamsters and mice have been shown to generate higher levels of reactive oxygen species (ROS), produce less ATP, and are therefore likely to have a reduced capacity to adequately support a dynamic process such as preimplantation development [26]. If a similar situation exists in humans, an increase in mitochondrial number may be necessary in the embryos of older women, in order for sufficient ATP levels to be maintained.

A decline in ATP synthetic capability with age could be related to an accumulation of mutations in the mitochondrial genome. An increase in the mtDNA content of human preimplantation embryos in response to mutation has previously been documented [27]. The location of the mtDNA in close proximity to ROS generated by the respiratory chain, coupled with a lack of histones and inferior DNA repair mechanisms, leaves the mitochondrial genome particularly vulnerable to mutation [8, 24]. In theory, the longer the oocyte remains in the ovary prior to fertilization, the greater the opportunity for mtDNA mutation to occur. Several studies have shown a reduction in mitochondrial gene expression in oocytes that fail to fertilize after exposure to sperm and in embryos that undergo developmental arrest. An increase in the incidence of the common mitochondrial 4977 bp deletion, associated with ageing in various tissues, has also been noted in human oocytes [24, 28, 29]. However, in the current study, sequencing of the entire mitochondrial genome using NGS failed to detect an obvious increase in mutation load in embryos with high mtDNA levels. This finding argues against the possibility that mitochondrial mutation is driving replication of the organelle in embryos from older women.

It may be that high mtDNA levels are indeed indicative of compromised mitochondria, but that the underlying defects are unrelated to alterations in the DNA sequence. Alternatively, elevated quantities of mtDNA might be associated with increased metabolic requirements of the embryo, rather than organelles of suboptimal function. It is possible that embryos produced by older oocytes are under some form of stress and therefore have larger energy requirements. Functional experiments will be required to address these questions. Whatever the underlying basis, the current study has unequivocally demonstrated that female reproductive aging is associated with changes in the mtDNA content at the blastocyst stage.

Aneuploidy affects more than half of all human preimplantation embryos and is believed to be the most important cause of early embryonic demise [18]. The majority of chromosome abnormalities are derived from errors occurring during oogenesis (meiotic, female origin), but chromosome malsegregation is also common during the first few embryonic cell divisions following fertilization (mitotic). Despite their frequency and clinical importance, the reasons for the high levels of meiotic and mitotic errors are still not fully understood.

As well as undergoing mtDNA quantification, all embryos analyzed during this study had previously been tested for aneuploidy as part of routine PGD or PGS using a well-validated comprehensive chromosome screening method [30, 31]. A comparison of the cytogenetic (aCGH) and mitochondrial (real-time PCR) data produced demonstrated that, on average, biopsy specimens derived from aneuploid blastocysts contained significantly greater amounts of mtDNA than samples from embryos that were euploid (P=0.025). These findings were confirmed using an alternative method (NGS) to assess an independent group of embryos. Importantly, the rise in mtDNA copy number seen in chromosomally abnormal embryos was additional to the association with female age, such that aneuploid blastocysts tended to have higher levels of mtDNA compared to chromosomally normal embryos derived from women of the same age.

It is plausible that variation in the quantity or functionality of mtDNA/mitochondria could have a direct effect on the accuracy of chromosome segregation. Mitochondrial metabolism factors, including ATP and the pyruvate dehydrogenase complex are essential for correct oocyte spindle assembly and chromosome alignment [32-34]. Furthermore, examination of oocytes from diabetic mice has demonstrated that damaged mitochondria are associated with aneuploidy. It is known that mitochondria are redistributed to spindles and microtubule organizing centers during cell division [35], presumably to ensure that the energy requirements of spindle formation and chromosome movement are satisfied. A link between mitochondrial distribution within the oocyte and chromosome congression on the meiotic spindle has been proposed [36]. Furthermore, it has been shown that embryos with high levels of chromosomal mosaicism, a consequence of errors occurring during the mitotic divisions following fertilization, frequently contain mitochondria with low membrane potential [37].

It is unclear at this time whether aneuploidy in embryos with increased quantities of mtDNA is a direct consequence of deficiencies affecting the organelle, disrupting ATP production or other key functions, or whether altered mitochondrial number and aneuploidy are independent, downstream consequences of another issue, currently undefined, affecting the embryo or the oocyte. It is important to note that although the increased quantities of mtDNA associated with age and aneuploidy were only seen in blastocysts, the trigger for expansion may already exist in oocytes prior to fertilization. Most of the aneuploidies observed in blastocysts are a consequence of errors occurring during female meiosis [30, 38], suggesting that factors that predispose to meiotic aneuploidy in oocytes might also have an effect on mtDNA replication during later embryonic stages.

mtDNA and Blastocyst Implantation Potential

In order to improve the efficiency of assisted reproductive treatments, superior methods for the identification of viable embryos are urgently required. The screening of embryos for cytogenetic abnormalities prior to transfer to the uterus allows the main cause of embryonic failure (i.e. aneuploidy) to be avoided. However, even the transfer of a morphologically ‘perfect’ embryo, which is additionally considered chromosomally normal following analysis of biopsied cells, cannot guarantee the initiation of a successful pregnancy (only about two thirds of such embryos actually produce a child). It is clear that additional elements play a role in embryo viability. Important factors might conceivably include mitochondrial number/capacity and accompanying effects on ATP content and/or metabolic activity [17]. As part of this investigation, the levels of mtDNA were retrospectively assessed in euploid cleavage and blastocyst stage embryos that had been transferred to the uterus following PGD or PGS and for which the clinical outcome was known.

The levels of mtDNA observed in biopsied cells were lower on average for blastocysts capable of establishing a clinical pregnancy compared to those that failed to implant after transfer (P=0.007). This relationship was initially identified using quantitative PCR, but was subsequently verified using NGS. The association between mtDNA quantity and ability to produce a pregnancy was only clearly observed in embryo samples taken at the blastocyst stage of development. The increases in mtDNA content associated with loss of embryo viability were more dramatic than those related to age or aneuploidy.

Analysis of the mtDNA content data allowed the establishment of a threshold above which implantation of a chromosomally normal blastocyst was never observed. This cut-off remained valid regardless of other considerations such as embryo morphology or the clinic where the patients were receiving treatment. Approximately one-third of non-viable blastocysts had mtDNA levels above the threshold, suggesting that this factor represents an indicator of lethally compromised embryos, second only to aneuploidy in terms of prevalence and clinical importance. In order to confirm the predictive power of mtDNA measurement, an independent series of blastocysts were blindly assessed in a prospective manner. Once again, all euploid blastocysts with mtDNA levels above the threshold failed to implant (100%). Those with quantities of mtDNA in the normal range displayed a 59% implantation rate, which contrasts to 38% for the group as a whole.

The failure to detect a clear association between mtDNA levels and implantation potential for cleavage stage embryos suggests that the elevated quantities of mtDNA seen in a subset on non-viable blastocysts are a consequence of an expansion that occurs after day-3 post-fertilization. An up-regulation in the expression of mtDNA replication factors is known to occur at the blastocyst stage, and this is generally considered to coincide with the first significant wave of mtDNA synthesis [3]. This may also be the time when excessive increases in mtDNA levels occur in some non-viable embryos.

Abnormally high levels of mtDNA at the blastocyst stage may be symptomatic of some form of stress that results in elevated energy requirements. This possibility would be consistent with the ‘quiet embryo hypothesis’, proposed by Leese, which suggests that viable embryos have relatively lower or ‘quiet’ metabolism, whereas those under stress, and of reduced developmental potential, tend to be more metabolically active [39].

It is of note that blastocysts with high mtDNA quantities, incapable of producing a viable pregnancy, were mostly generated by women aged 38 years or older. This observation is not surprising considering that an increase in mtDNA in relation to advancing female age had been observed. The association between female age and diminished embryo viability is well established and known to be primarily due to aneuploidy [40]. However, our findings suggest mitochondria represent an important additional factor.

### SUMMARY

These results demonstrate a clear association between mtDNA quantity and the ability of a human embryo to implant in the uterus. Specifically, the results provided above show that the quantity of mtDNA was significantly higher in embryos from older women (P=0.003). Additionally, mtDNA levels were elevated in aneuploid embryos, independent of age (P=0.025). Assessment of clinical outcomes after transfer of euploid embryos to the uterus revealed that blastocysts that successfully implanted tended to contain lower mtDNA quantities than those failing to implant (P=0.007). Importantly, an mtDNA quantity threshold was established, above which implantation was never observed. Subsequently, the predictive value of this threshold was confirmed in an independent blinded prospective study, indicating that abnormal mtDNA levels are present in 30% of non-implanting euploid embryos, but are not seen in embryos forming a viable pregnancy. NGS did not reveal any increase in mutation in blastocysts with elevated mtDNA levels. The results of this study suggest that increased mtDNA may be related to elevated metabolism and are associated with reduced viability. Importantly, the findings suggest a potential role for mitochondria in female reproductive aging and the genesis of aneuploidy. Of clinical significance, we propose that mtDNA content represents a novel biomarker with potential value for in vitro fertilization (IVF) treatment, revealing chromosomally normal blastocysts incapable of producing a viable pregnancy.

These results establish an mtDNA threshold above which implantation failure was 100%. The data obtained suggest that embryo deficiencies associated with elevated mtDNA explain up to one-third of implantation failures affecting blastocysts diagnosed euploid. The defined mtDNA threshold does not appear to be altered by variation in the processes used by different fertility clinics, indicating that evaluation of mtDNA in embryos could form the basis of a simple, inexpensive and widely applicable clinical test.

Relationships between mtDNA content, female age and embryo chromosomal status were also demonstrated. The possibility that mtDNA content has a direct influence on embryo viability and the potential for a causal relationship with aneuploidy, and other factors related to reproductive senescence warrant further investigation.

### Example 8: Quantification of Mitochondrial DNA by Real Time PCR

### Reference DNA Preparation

A reference DNA is selected from a single Sureplex product (amplified DNA from Trophectoderm sample) or it can be prepared by mixing 10-20 μl of various different Sureplex products. It is advisable to prepare 10-12 aliquots of 15 μl of the reference DNA and store them in the −80° C. freezer. One of these aliquots can be used for each of the real-time PCR plates.

TABLE 4
Materials.

| | | Catalog | Storage | |
|---|---|---|---|---|
| Item | Company | Number | conditions | |
| Taqman Universal | Life Technologies | 4440040 | 4° | C. |
| MMIX II- no UNG | | | | |
| Custom Taqman Gene | Life Technologies | 4332078 | −20° | C. |
| expression Assays- | | | | |
| mtDNA primers | | | | |
| Custom Taqman Gene | Life Technologies | 4332078 | −20° | C. |
| expression Assays- | | | | |
| Alu primers | | | | |
| Custom-custom | Life Technologies | order | −20° | C. |
| Oligios-mtDNAMjrArc | | through | | |
| | | customer | | |
| | | service | | |

### Primer Sequences:

mtDNA Assay
MTDNA_16S_F
(SEQ ID NO: 2)
GGTGATAGCTGGTTGTCCAAGAT
MTDNA_16S_R
(SEQ ID NO: 3)
CCTACTATGGGTGTTAAATTTTTTACTCTCTC
MTDNA_16S_M
(SEQ ID NO: 4)
AATTTAACTGTTAGTCCAAAGAG
FAM-MGBNFQ
Medium
ALU Assay
YB8-ALU-S68_F
(SEQ ID NO: 5)
GTCAGGAGATCGAGACCATCCT
YB8-ALU-S68_R
(SEQ ID NO: 6)
AGTGGCGCAATCTCGGC
YB8-ALU-S68_M
(SEQ ID NO: 7)
AGCTACTCGGGAGGCTGAGGCAGGA
FAM-MGBNFQ
Medium
mtMajArc
mtMajArc_F
(SEQ ID NO: 8)
CTGTTCCCCAACCTTTTCCT
mtMajArc_R
(SEQ ID NO: 9)
CCATGATTGTGAGGGGTAGG
mtMajArc_M
(SEQ ID NO: 10)
GACCCCCTAACAACCCCC
NED-NFQ
Medium

### Sample Preparation for Real-Time PCR

For each 96-well plate a positive (reference DNA) and a negative (nuclease-free water) controls are analyzed, along with the trophectoderm (TE) samples. All reactions are performed in triplicate. A mastermix of water and sample, reference or negative control is prepared in the following way.

| | Volume (μl)/ | Master-mix for |
|---|---|---|
| Component | Reaction | 4 tubes |
| Nuclease free water | 8 | 8 × 4 = 32 μl |
| Sample DNA | 1 | 1 × 4 = 4 μl |
| Total volume | 9 | 36 μl |

* Negative control preparation (one well only) - Nuclease-free water: 32 μl

As the real-time PCR involves the analysis of 3 TaqMan assays, the above mastermix is prepared separately for each of the samples and each of the 3 TaqMan assays.

Once these aliquots are prepared they are vortexed, centrifuged, and stored at 4° C. until they are to be used.

The aliquots should be prepared in a PCR enclosure in the main lab. Ideally the same pipettes should be used for all the preparation of aliquots, real-time PCR and sample loading on the plate.

### Real-Time PCR Protocol

Three TaqMan assays are assessed: Mitochondria (2 TaqMan assays: mtDNA and MajArc), and ALU (1 TaqMan Assay). The mastermixes for these are prepared and aliquoted in the plate in the single cell room.

### Master-Mix 1: ALU

No. of tubes: 34 (this is for 96 wells)

| | Volume (μl)/ | Master-mix for |
|---|---|---|
| Component | Reaction | 37 tubes |
| TaqMan Universal | 10 | 10 × 37 = 370 μl |
| Master-mix II | | |
| (2X)/no UNG | | |
| TaqMan Assay | 1 | 1 × 37 = 37 μl |
| (20X)/ALU | | |
| Total volume | 11 | 407 μl |

Aliquot 11 μl into the first 33 wells of a 96-well plate (A2-A11, B1-C9)

### Master-Mix 2: Mitochondria/mtDNA TaqMan Assay

No. of tubes: 34 (this is for 96 wells)

| | Volume (μl)/ | Master-mix for |
|---|---|---|
| Component | Reaction | 37 tubes |
| TaqMan Universal | 10 | 10 × 37 = 370 μl |
| Master-mix II | | |
| (2X)/no UNG | | |
| TaqMan Assay | 1 | 1 × 37 = 37 μl |
| (20X)/MTDNA | | |
| Total volume | 11 | 407 μl |

Aliquot 11 μl into the next 31 wells of a 96-well plate (C10-F4)

### Master-Mix 3: Mitochondria/ MajArc

No. of tubes: 34 (this is for 96 wells)

| | Volume (μl)/ | Master-mix for |
|---|---|---|
| Component | Reaction | 37 tubes |
| TaqMan Universal | 10 | 10 × 37 = 370 μl |
| Master-mix II | | |
| (2X)/no UNG | | |
| TaqMan Assay | 1 | 1 × 37 = 37 μl |
| (20X)/MajArc | | |
| Total volume | 11 | 407 μl |

Aliquot 11 μl into the next 32 wells of a 96-well plate (F5-G12, H2-H12)

Once all master-mixes have been placed in the wells of the plate, take the plate in the hood the main lab and aliquot the appropriate amount of DNA samples, i.e. 9 μl

Vortex and spin.

The thermal cycling conditions are as follows:

| Step | | | | |
|---|---|---|---|---|
| | Polymerase | PCR | | |
| | activation | Cycles (35) | | |
| | HOLD | Denature | Anneal | Extend |

| | | | | | | | | |
|---|---|---|---|---|---|---|---|---|
| Time | 10 | min | 15 | sec | 15 | sec | 1 | min |
| Temp | 95° | C. | 95° | C. | 55° | C. | 60° | C. |

### Analysis of the Results

### First Plate

The Delta Cts (ΔC_{T}) were calculated for the reference DNA and TE samples and for both mitochondrial TaqMan assays. The mean Ct value of the Alu TaqMan assay was deducted from the mean Ct values of each of the mtDNA and MajArc mitochondrial TaqMan assays. The mtDNA for each of the analyzed samples:

Delta Ct(ΔC_{T})=mtDNA mean Ct−Alu mean Ct

Delta Ct(ΔC_{T})=MajArc mean Ct−Alu mean Ct

The relative mitochondrial DNA value was calculated for the reference DNA and each of the TE samples, and for each of the two mitochondrial TaqMan assays by the equation 2^{-Delta Ct} (2^{-ΔCT}).

The threshold for the mtDNA primer was 0.00005, and that for the MajArc was 0.000024. Samples with higher values had lower implantation potential. In order to call a sample high, the values obtained for both TaqMan assays were over the set thresholds.

### All Other Plates

The Delta Cts (ΔC_{T}) for the reference DNA and TE samples and for both mitochondrial TaqMan assays were calculated. The mean Ct value of the Alu TaqMan assay was deducted from the mean Ct values of each of the mtDNA and MajArc mitochondrial TaqMan assays.

The mtDNA for each of the analyzed samples was calculated as follows:

Delta Ct(ΔC_{T})=mtDNA mean Ct−Alu mean Ct

The MajArc for each of the analyzed samples was calculated as follows:

Delta Ct(ΔC_{T})=MajArc mean Ct−Alu mean Ct

Values were normalized via the calculation of the Delta Delta Ct (ΔΔC_{T}) to ensure that the samples in the rest of the plates behaved the same way with those in the first plate.

To do this the normalization factor was calculated by deducting the reference DNA Delta Ct (ΔC_{T}) value obtained in the current plate from the Delta Ct (ΔC_{T}) value of the reference DNA in the first plate. The resulting value was the normalization factor. This was done for both mitochondrial TaqMan assays.

To calculate the Delta Delta Ct (ΔΔC_{T}), the normalization factor value was added to the Delta Ct (ΔC_{T}) values obtained for the analyzed TE samples. This was done for both mitochondrial TaqMan assays.

As previously the relative mtDNA value was calculated via the equation 2^{-Delta Delta Ct} (2^{-ΔΔCT}).

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

### REFERENCES

1. May-Panloup P, Chrétien M F, Jacques C, Vasseur C, Malthièry Y, et al. (2005) Low oocyte mitochondrial DNA content in ovarian insufficiency. Hum Reprod 20: 593-597.
2. Dumollard R, Carroll J, Duchen M R, Campbell K, Swann K (2009) Mitochondrial function and redox state in mammalian embryos. Semin Cell Dev Biol 20: 346-353.
3. St John J C, Facucho-Oliveira J, Jiang Y, Kelly R, Salah R (2010) Mitochondrial DNA transmission, replication and inheritance: a journey from the gamete through the embryo and into offspring and embryonic stem cells. Hum Reprod Update 16: 488-509.
4. Bentov Y, Casper R F (2013) The aging oocyte—can mitochondrial function be improved? Fertil Steril 99: 18-22.
5. Tilly J L, Sinclair D A (2013) Germline energetics, aging, and female infertility. Cell Metab 17: 838-850.
6. Anderson S, Bankier A T, Barrell B G, de Bruijn M H, Coulson A R, et al. (1981) Sequence and organization of the human mitochondrial genome. Nature 290: 457-465.
7. Palmer C S, Osellame L D, Stojanovski D, Ryan M T (2011) The regulation of mitochondrial morphology: intricate mechanisms and dynamic machinery. Cell Signal 23: 1534-1545.
8. Eichenlaub-Ritter U, Wieczorek M, Luke S, Seidel T (2011) Age related changes in mitochondrial function and new approaches to study redox regulation in mammalian oocytes in response to age or maturation conditions. Mitochondrion 11: 783-796.
9. Chen X, Prosser R, Simonetti S, Sadlock J, Jagiello G, et al. (1995) Rearranged mitochondrial genomes are present in human oocytes. Am J Hum Genet 57: 239-247.
10. Motta P M, Nottola S A, Makabe S, Heyn R (2000). Mitochondrial morphology in human fetal and adult germ cells. Hum Reprod 15: 129-147.
11. Cummins J M (2000) Fertilization and elimination of the paternal mitochondrial genome. Hum. Reprod 15: 92-101.
12. Steuerwald N, Barritt J A, Adler R, Malter H, Schimmel T, et al. (2000) Quantification of mtDNA in single oocytes, polar bodies and subcellular components by real-time rapid cycle fluorescence monitored PCR. Zygote 8: 209-215.
13. Reynier P, May-Panloup P, Chrétien M F, Morgan C J, Jean M, et al. (2001) Mitochondrial DNA content affects the fertilizability of human oocytes. Mol Hum Reprod 7: 425-429.
14. Barritt J A, Kokot M, Cohen J, Steuerwald N, Brenner C A (2002) Quantification of human ooplasmic mitochondria. Reprod Biomed Online 4: 243-247.
15. Lin D P, Huang C C, Wu H M, Cheng T C, Chen C I, et al. (2004) Comparison of mitochondrial DNA contents in human embryos with good or poor morphology at the 8-cell stage. Fertil Steril 81: 73-79.
16. Chan C C, Liu V W, Lau E Y, Yeung W S, Ng E H, et al. (2005) Mitochondrial DNA content and 4977 bp deletion in unfertilized oocytes. Mol Hum Reprod 11: 843-846.
17. Van Blerkom J, Davis P W, Lee J (1995) ATP content of human oocytes and developmental potential and outcome after in-vitro fertilization and embryo transfer. Hum Reprod 10: 415-424.
18. Fragouli E, Alfarawati S, Spath K, Jaroudi S, Sarasa J, et al. (2013). The origin and impact of embryonic aneuploidy. Hum Genet 132: 1001-1013.
19. Van Blerkom J (2011) Mitochondrial function in the human oocyte and embryo and their role in developmental competence. Mitochondrion 11: 797-813.
20. Houghton F (2006) Energy metabolism of the inner cell mass and trophectoderm of the mouse blastocyst. Differentiation 74: 11-18.
21. Barritt J A, Cohen J, Brenner C A (2000) Mitochondrial DNA point mutation in human oocytes is associated with maternal age. Reprod Biomed Online 1: 96-100.
22. Konstantinidis M, Alfarawati S, Hurd D, Paolucci M, Shovelton J, et al. (2014) Simultaneous assessment of aneuploidy, polymorphisms, and mitochondrial DNA content in human polar bodies and embryos with the use of a novel microarray platform. Fertil Steril: in press.
23. Narita A (1995) Endogenous factors affecting sterility in oocytes of aged animals. Jap J Fertil Steril 40: 57-65.
24. Duran H E, Simsek-Duran F, Oehninger S C, Jones H W Jr, Castora (2011) The association of reproductive senescence with mitochondrial quantity, function, and DNA integrity in human oocytes at different stages of maturation. Fertil Steril 96: 384-388.
25. Piko L, Taylor K D (1987) Amounts of mitochondrial DNA and abundance of some mitochondrial gene transcripts in early mouse embryos. Dev Biol 123: 364-374.
26. Simsek-Duran F, Li F, Ford W, Swanson R J, Jones H W Jr, et al. (2009) The effect of aging on the metabolic function and structure of mitochondria in hamster oocytes. FASEB J 855: 10.
27. Monnot S, Samuels D C, Hesters L, Frydman N, Gigarel N, et al. (2013) Mutation dependance of the mitochondrial DNA copy number in the first stages of human embryogenesis. Hum Mol Genet 22: 1867-1872.
28. Hsieh R H, Au H K, Yeh T S, Chang S J, Cheng Y F, et al. (2004) Decreased expression of mitochondrial genes in human unfertilized oocytes and arrested embryos. Fertil Steril 81: 912-918.
29. Hsieh R H, Tsai N M, Au H K, Chang S J, Wei Y H, et al. (2002) Multiple rearrangements of mitochondrial DNA in unfertilized human oocytes. Fertil Steril 77: 1012-1017.
30. Fragouli E, Alfarawati S, Daphnis D D, Goodall N N, Mania A, et al. (2011) Cytogenetic analysis of human blastocysts with the use of FISH, CGH and aCGH: scientific data and technical evaluation. Hum Reprod 26:480-490
31. Wells D, Kaur K, Grifo J, Glassner M, Taylor J C, et al. (2014) Clinical utilisation of a rapid low-pass whole genome sequencing technique for the diagnosis of aneuploidy in human embryos prior to implantation. J Med Genet 51: 553-562.
32. Zhang X, Wu X Q, Lu S, Guo Y L, Ma X (2006) Deficit of mitochondria-derived ATP during oxidative stress impairs mouse MII oocyte spindles. Cell Res 16: 841-850.
33. Choi W J, Banerjee J, Falcone T, Bena J, Agarwal A, et al. (2007) Oxidative stress and tumor necrosis factor-induced alterations in metaphase II mouse oocyte spindle structure. Fertil Steril 88:1220-1231.
34. Johnson M T, Freeman E A, Gardner D K, Hunt P A (2007) Oxidative metabolism of pyruvate is required for meiotic maturation of murine oocytes in vivo. Biol Reprod 77: 2-8.
35. Yu Y, Dumollard R, Rossbach A, Lai F A, Swann K (2010) Redistribution of mitochondria leads to bursts of ATP production during spontaneous mouse oocyte maturation. Journal of Cellular Physiology 224: 672-680.
36. Wang Q, Ratchford A M, Chi MINI, Schoeller E, Frolova A, et al. (2009) Maternal diabetes causes mitochondrial dysfunction and meiotic defects in murine oocytes. Mol Endocrinol 23: 1603-1612.
37. Wilding M, De Placido G, DeMatteo L, Marino M, Alviggi C, et al. (2003) Chaotic mosaicism in human preimplantation embryos is correlated with a low mitochondrial membrane potential, Fertil Steril 79: 340-346.
38. Capalbo A, Wright G, Elliott T, Ubaldi F M, Rienzi L et al. (2013) FISH reanalysis of inner cell mass and trophectoderm samples of previously array-CGH screened blastocysts shows high accuracy of diagnosis and no major diagnostic impact of mosaicism at the blastocyst stage. Hum Reprod 28: 2298-2307.
39. Leese H J (2002) Quiet please, do not disturb: a hypothesis of embryo metabolism and viability. Bioessays 24: 845-849.
40. Harton G L, Munné S, Surrey M, Grifo J, Kaplan B, McCulloh D H, et al. (2013) Diminished effect of maternal age on implantation after preimplantation genetic diagnosis with array comparative genomic hybridization. Fertil Steril 100: 1695-1670
41. Magli M C, Montag M, Koster M, Muzi L, Geraedts J, et al. (2011) Polar body array CGH for prediction of the status of the corresponding oocyte. Part II: technical aspects. Hum Reprod 26:3181-3185.
42. Gutierrez-Mateo C, Colls P, Sanchez-Garcia J, Escudero T, Prates R, et al. (2011) Validation of microarray comparative genomic hybridization for comprehensive chromosome analysis of embryos. Fertil Steril 95:953-958.
43. Christopikou D, Tsorva E, Economou K, Shelley P, Davies S, et al. (2013) Polar body analysis by array comparative genomic hybridization accurately predicts aneuploidies of maternal meiotic origin in cleavage stage embryos of women of advanced maternal age. Hum Reprod 28: 1426-1434.
44. Mertzanidou A, Spits C, Nguyen H T, Van de Velde H, Sermon K (2013) Evolution of aneuploidy up to Day 4 of human preimplantation development. Hum Reprod 28: 1716-1724.
45. Fregel R, Almeida M, Betancor E, Suarez N M, Pestano J (2011) Reliable nuclear and mitochondrial DNA quantification for low copy number and degraded forensic samples. Forensic Science International: Genetics Supplement Series 3: e303-e304.
46. Li H, Durbin R (2009) Fast and accurate short read alignment with Burrows-Wheeler transform. Bioinformatics 25: 1754-1760.
47. Raczy C, Petrovski R, Saunders C T, Chorny I, Kruglyak S et al. (2013) Isaac: ultra-fast whole-genome secondary analysis on Illumina sequencing platforms. Bioinformatics 29:2041-2043
48. Quinlan A R, Hall I M (2010) BEDTools: A flexible suite of utilities for comparing genomic features, Bioinformatics 26: 841-842.
49. Li H, Handsaker B, Wysoker A, Fennell T, Ruan J, et al. (2009) The Sequence alignment/map (SAM) format and SAMtools. Bioinformatics 25: 2078-2079.
50. Baslan T, Kendall J, Rodgers L, Cox H, Riggs M, et al. (2012) Genome-wide copy number analysis of single cells. Nat Protoc 7: 1024-1041.
51. Zhidkov I, Nagar T, Mishmar D, Rubin E (2011) MitoBamAnnotator: A web-based tool for detecting and annotating heteroplasmy in human mitochondrial DNA sequences. Mitochondrion 11: 924-928.
52. Schmittgen T D, Livak K J (2008) Analyzing real-time PCR data by the comparative CT method. Nat Protoc 3: 1101-1108.

What is claimed is:

1. A method for determining the relative quantity of mitochondrial DNA in an embryo, the method comprising:
providing a DNA sample obtained from the embryo;
determining an amount of mitochondrial DNA (mtDNA) in the DNA sample;
determining an amount of a reference DNA in the DNA sample; and
comparing the amount of mtDNA to the amount of reference DNA to determine a relative quantity of mtDNA in the embryo.

2. The method of claim 1, wherein the relative quantity of mtDNA in the embryo is indicative of the implantation potential of the embryo.

3. The method of claim 1, wherein the determining the amount of mtDNA in the DNA sample and the determining the amount of a reference DNA in the DNA sample is preformed using quantitative PCR.

4. The method of claim 3, wherein the quantitative PCR comprises real time PCR.

5. The method of claim 1, wherein a DNA sample is obtained from the embryo 1, 2, 3, 4, 5, 6 or 7 days posit implantation, or 1-3 days, 2-4 days, 3-5 days, 5-7 days, 1-7 days, or 4-7 days post fertilization.

6. The method of claim 1, wherein the embryo is a euploid embryo.

7. The method of claim 1, wherein the determining the amount of mtDNA in the DNA sample comprises amplifying the mtDNA by contacting the DNA sample with a primer pair comprising SEQ ID NOs: 2 and 3 targeting a sequence transcribing 16S to produce a 16S amplicon or a primer pair comprising SEQ ID NOs: 8 and 9 targeting a sequence encoding NADH-ubiquinone oxidoreductase chain 4 (MT-ND4) to produce a MT-ND4 amplicon.

8. The method of claim 7, further comprising detecting the 16S amplicon by contacting the DNA sample with a probe comprising SEQ ID NO: 4.

9. The method of claim 7, further comprising detecting the MT-ND4 amplicon by contacting the DNA sample with a probe comprising SEQ ID NO: 10.

10. The method of claim 1, wherein the determining the amount of a reference DNA in the DNA sample comprises amplifying a reference DNA sequence by contacting the DNA sample with a primer pair comprising SEQ ID NOs: 5 and 6 targeting an Alu sequence to produce an Alu amplicon.

11. The method of claim 10, further comprising detecting the Alu amplicon by contacting the DNA sample with a probe comprising SEQ ID NO: 7.

12. The method of claim 1, further comprising:
comparing the relative quantity of mtDNA in the euploid embryo to an implantation potential threshold;
wherein a relative quantity of mtDNA in the embryo below the implantation potential threshold is indicative of a favorable implantation potential of the euploid embryo and a relative quantity of mtDNA in the embryo exceeding the implantation potential threshold is indicative of an unfavorable implantation potential of the euploid embryo.

13. A method for selecting an embryo for implantation, the method comprising:
determining the relative amount of mitochondrial DNA in an embryo sample compared to a reference chromosomal nucleic acid sequence in the embryo, wherein determining comprises preparing a reaction mix comprising
i) a nucleic acid sample from an embryo;
ii) a first oligonucleotide primer pair directed against a first mitochondrial DNA sequence; and
iii) a reference oligonucleotide primer pair directed against a reference target nucleic acid sequence;
amplifying the reaction mixture to produce a first mitochondrial DNA sequence product and a reference target nucleic acid sequence product;
assessing the amount of the amplified first mitochondrial DNA sequence product compared with the amount of the amplified reference target nucleic acid sequence product; and
selecting an embryo for implantation when the measured amount of the amplified first mitochondrial DNA sequence product relative to the amplified reference target nucleic acid sequence product are below an implantation potential threshold value.

14. The method of claim 13, wherein the reaction mix further comprises a second synthetic oligonucleotide primer pair directed against a second mitochondrial DNA sequence.

15. The method of claim 13, further comprising:
amplifying the reaction mixture to produce a first mitochondrial DNA sequence product, a second mitochondrial DNA sequence product; and a reference target nucleic acid sequence product, and measuring the amount of each amplified product;
comparing the measured amount of the amplified first mitochondrial DNA sequence product and the amplified second mitochondrial DNA sequence product with the amplified reference target nucleic acid sequence product; and
selecting an embryo for implantation when the measured amount of the amplified first mitochondrial DNA sequence product and the amplified second mitochondrial DNA sequence product relative to the amplified reference target nucleic acid sequence product are below an implantation potential threshold value.

16. The method of claim 13, further comprising a third synthetic oligonucleotide primer pair directed against a third mitochondrial DNA sequence.

17. A method for selecting an embryo for implantation, the method comprising:
measuring the relative amount of mitochondrial DNA in an embryo sample compared to a reference nucleic acid sample, wherein determining comprises preparing a reaction mix comprising
i) a nucleic acid sample from an embryo;
ii) a first synthetic oligonucleotide primer pair directed against a first mitochondrial DNA sequence;
iii) a second synthetic oligonucleotide primer pair directed against a second mitochondrial DNA sequence; and
iv) a reference synthetic oligonucleotide primer pair directed against a of reference target nucleic acid sequence;
amplifying the reaction mixture to produce a first mitochondrial DNA sequence product, a second mitochondrial DNA sequence product; and a reference target nucleic acid sequence product, and measuring the amount of each amplified product;
comparing the measured amount of the first mitochondrial DNA sequence product and the second mitochondrial DNA sequence product with the reference target nucleic acid sequence product; and
selecting an embryo for implantation when the measured amount of the first mitochondrial DNA sequence product and the second mitochondrial DNA sequence product relative to the reference target nucleic acid sequence product are below an implantation potential threshold value.

18. The method of claim 17, further comprising:
identifying embryos as not being suitable for implantation when the amount of the first mitochondrial DNA sequence product and the second mitochondrial DNA sequence product relative to the reference target nucleic acid sequence product are above an implantation potential threshold value.

19. The method of claim 18, wherein the first or second synthetic oligonucleotide primer pair is directed against a mitochondrial DNA sequence encoding a 12S RNA a mitochondrial DNA sequence encoding a 16S RNA, a mitochondrial DNA sequence encoding NADH dehydrogenase subunit 1 (MT-ND1), a mitochondrial DNA sequence NADH dehydrogenase subunit 1 (MT-ND1), a mitochondrial DNA sequence encoding NADH dehydrogenase subunit 2 (MT-ND2), a mitochondrial DNA sequence encoding NADH dehydrogenase subunit 3 (MT-ND3), a mitochondrial DNA sequence encoding NADH dehydrogenase subunit 4 (MT-ND4), a mitochondrial DNA sequence encoding NADH dehydrogenase subunit 5 (MT-ND5), a mitochondrial DNA sequence encoding Cytochrome b, mitochondrial Cytochrome c oxidase subunit 1, 2 or 3, a mitochondrial DNA sequence encoding an ATP synthase.

20. The method of claim 19, wherein the first synthetic oligonucleotide primer pair is directed against the mitochondrial DNA sequence encoding the 16S rRNA gene sequence.

21. The method of claim 19, wherein the second synthetic oligonucleotide primer pair is directed against the mitochondrial DNA sequence encoding the NADH-ubiquinone oxidoreductase chain 4 (MT-ND4) gene sequence.

22. The method of claim 18, wherein the reference synthetic oligonucleotide primer pair is directed against the Alu sequence, the L1 sequence, glyceraldehyde 3-phosphate dehydrogenase (GAPDH), or β-actin (ActB).

23. The method of claim 22, wherein the reference synthetic oligonucleotide primer pair is directed against the Alu sequence.

24. The method of claim 20, wherein the first synthetic oligonucleotide primer pair comprises primer having at least 70% sequence identity to a nucleic acid sequence of SEQ ID NO: 2 and primer having at least 70% sequence identity to a nucleic acid sequence of SEQ ID NO: 3.

25. The method of claim 21, wherein the second synthetic oligonucleotide primer pair comprises primer having at least 70% sequence identity to a nucleic acid sequence of SEQ ID NO: 8 and primer having at least 70% sequence identity to a nucleic acid sequence of SEQ ID NO: 9.

26. The method of claim 23, wherein the reference synthetic oligonucleotide primer pair comprises primer having at least 70% sequence identity to a nucleic acid sequence of SEQ ID NO: 5 and primer having at least 70% sequence identity to a nucleic acid sequence of SEQ ID NO: 6.

27. The method of claim 17, wherein amplifying is preformed using a quantitative or semi-quantitative RT-PCR method.

28. A kit for determining the implantation potential of an embryo, comprising:
a first synthetic oligonucleotide primer pair directed against a first mitochondrial DNA sequence;
a second synthetic oligonucleotide primer pair directed against a of reference gene sequence.

29. The kit of claim 28, wherein the first synthetic oligonucleotide primer pair is directed a mitochondrial DNA sequence encoding a 12S RNA a mitochondrial DNA sequence encoding a 16S RNA, a mitochondrial DNA sequence encoding NADH dehydrogenase subunit 1 (MT-ND1), a mitochondrial DNA sequence NADH dehydrogenase subunit 1 (MT-ND1), a mitochondrial DNA sequence encoding NADH dehydrogenase subunit 2 (MT-ND2), a mitochondrial DNA sequence encoding NADH dehydrogenase subunit 3 (MT-ND3), a mitochondrial DNA sequence encoding NADH dehydrogenase subunit 4 (MT-ND4), a mitochondrial DNA sequence encoding NADH dehydrogenase subunit 5 (MT-ND5), a mitochondrial DNA sequence encoding Cytochrome b, mitochondrial Cytochrome c oxidase subunit 1, 2 or 3, a mitochondrial DNA sequence encoding an ATP synthase.

30. The kit of claim 29, wherein the first synthetic oligonucleotide primer pair is directed against the mitochondrial DNA sequence encoding the 16S rRNA gene sequence.

31. The kit of claim 30, wherein the first synthetic oligonucleotide primer pair comprises primer having at least 70% sequence identity to a nucleic acid sequence of SEQ ID NO: 2 and primer having at least 70% sequence identity to a nucleic acid sequence of SEQ ID NO: 3.

32. The kit of claim 30, further comprising a probe directed against the mitochondrial DNA sequence encoding the 16S rRNA, wherein the probe comprises SEQ ID NO: 4.

33. The kit of claim 29, wherein the first synthetic oligonucleotide primer pair is directed against the mitochondrial DNA sequence encoding the NADH-ubiquinone oxidoreductase chain 4 (MT-ND4) gene sequence.

34. The kit of claim 33, wherein the first synthetic oligonucleotide primer pair comprises primer having at least 70% sequence identity to a nucleic acid sequence of SEQ ID NO: 8 and primer having at least 70% sequence identity to a nucleic acid sequence of SEQ ID NO: 9.

35. The kit of claim 33, further comprising a probe directed against the mitochondrial DNA sequence encoding the MT-ND4, wherein the probe comprises SEQ ID NO: 10.

36. The kit of claim 28, wherein the reference synthetic oligonucleotide primer pair is directed against the Alu sequence, the L1 sequence, glyceraldehyde 3-phosphate dehydrogenase (GAPDH), or β-actin (ActB).

37. The kit of claim 36, wherein the reference synthetic oligonucleotide primer pair is directed against the Alu sequence.

38. The kit of claim 37, wherein the reference synthetic oligonucleotide primer pair comprises primer having at least 70% sequence identity to a nucleic acid sequence of SEQ ID NO: 5 and primer having at least 70% sequence identity to a nucleic acid sequence of SEQ ID NO: 6.

39. The kit of claim 36, further comprising a probe directed against the Alu sequence, wherein the probe comprises SEQ ID NO: 7.

40. The kit of claim 28, further comprising deoxynucleotides (dNTPs).

41. The kit of claim 28, further comprising a DNA polymerase.

42. The kit of claim 41, wherein the DNA polymerase is a thermostable DNA polymerase.

43. The kit of claim 42, wherein the thermostable DNA polymerases is a Taq DNA polymerase or an AmpliTaq® DNA polymerase.

44. The kit of claim 28, further comprising a buffer.

45. The kit of claim 44, wherein the buffer is a Tris-EDTA (TE) buffer.

46. The kit of claim 28, further comprising a positive control DNA sample and a negative control DNA sample.

47. The kit of claim 28, further comprising a third synthetic oligonucleotide primer pair directed against a second mitochondrial DNA sequence.

48. A method comprising:
determining the amount of mitochondrial DNA (mtDNA) in one or more DNA samples obtained from implanting euploid embryos;
determining the amount of mtDNA in one or more DNA samples obtained from non-implanting euploid embryos; and
calculating an implantation potential threshold value by comparing the amount of mtDNA from implanting euploid embryos with the amount of mtDNA obtained from the non-implanting euploid embryos.

49. The method of claim 48, wherein the determining the amount of mtDNA in a DNA sample is performed using quantitative PCR.

50. The method of claim 49, wherein the quantitative PCR comprises real time PCR.

51. The method of claim 48, wherein the determining the amount of mtDNA in the first DNA sample comprises:
amplifying the mtDNA in the sample by contacting the DNA sample with a primer pair comprising SEQ ID NOs: 2 and 3 targeting a sequence transcribing 16S to produce a 16S amplicon or a primer pair comprising SEQ ID NOs: 8 and 9 targeting a sequence encoding NADH-ubiquinone oxidoreductase chain 4 (MT-ND4) to produce a MT-ND4 amplicon; and
amplifying a reference DNA sequence by contacting the DNA sample with a primer pair comprising SEQ ID NOs: 5 and 6 targeting an Alu sequence to produce an Alu amplicon.

52. The method of claim 51, further comprising:
detecting the 16S amplicon by contacting the DNA sample with a probe comprising SEQ ID NO: 4 targeting a sequence within the 16S amplicon; and
detecting the Alu amplicon by contacting the DNA sample with a probe comprising SEQ ID NO: 7 targeting a sequence within the Alu amplicon.

53. The method of claim 51, further comprising:
detecting the MT-ND4 amplicon by contacting the DNA sample with a probe comprising SEQ ID NO: 10 targeting a sequence within the MT-ND4 amplicon; and
detecting the Alu amplicon by contacting the DNA sample with a probe comprising SEQ ID NO: 7 targeting a sequence within the Alu amplicon.

54. A method for determining the implantation potential of an embryo, the method comprising:
determining the relative amount of mitochondrial DNA in an embryo sample compared to a reference nucleic acid sample, wherein determining comprises preparing a reaction mix comprising
i) a nucleic acid sample from an embryo;
ii) a first synthetic oligonucleotide primer pair directed against a first mitochondrial DNA sequence; and
iii) a reference synthetic oligonucleotide primer pair directed against a of reference target nucleic acid sequence;
amplifying the reaction mixture to produce a first mitochondrial DNA sequence product and a reference chromosomal target nucleic acid sequence product; and
assessing the amount of the amplified first mitochondrial DNA sequence product compared with the amount of the amplified reference target nucleic acid sequence product, wherein the relative quantity of mtDNA in the embryo is indicative of the implantation potential of the embryo.

55. The method of claim 54, wherein
determining the relative amount of mitochondrial DNA further comprises: iv) a second synthetic oligonucleotide primer pair directed against a second mitochondrial DNA sequence; and
the amplifying step further comprises amplifying the reaction mixture to produce a second mitochondrial DNA sequence product; and
the assessing step further comprises assessing the amount of the amplified second mitochondrial DNA sequence product with the amount of the amplified reference target nucleic acid sequence product,
wherein the relative quantity of the first and the second mitochondrial DNA sequence products are indicative of the implantation potential of the embryo.
